(19) United States
(12) Patent Application Publication
(10) Pub. No.: US 2008/0076974 A1
(43) Pub. Date: Mar. 27, 2008

(54) Biological information detection sensor device

(75) Inventors: Shunpei Yamazaki, Setagaya (JP); Yasuyuki Arai, Atsugi (JP); Miho Komori, Isehara (JP)

Correspondence Address: ERIC ROBINSON, PMB 955, 21010 SOUTHBANK ST., POTOMAC FALLS, VA, 20165, US

(73) Assignee: Semiconductor Energy Laboratory Co., Ltd., Atsugi-shi (JP)

(21) Appl. No.: 11/785,963

(22) Filed: Apr. 23, 2007

(30) Foreign Application Priority Data
Apr. 28, 2006 (JP) 2006-124784

(51) Int. Cl. A61B 5/00; A61B 5/04

(52) U.S. Cl. 600/300; 600/391

(57) ABSTRACT

It is an object of the present invention to provide a biological information detection sensor system which can be easily replaced. The present invention relates to a biological information detection sensor system having an adhesive tape including an adhesive surface which is provided with an adhesive material and a surface which is not provided with the adhesive material; a biological information detection sensor having an ID tag in which personal information is stored and a sensor, which is attached to an adhesive surface side of the adhesive tape; and an antenna which is connected to the biological information detection sensor and is pulled from the adhesive surface of the adhesive tape to the surface thereof through a groove provided in the adhesive tape. Accordingly, even if the biological information detection sensor gets dirty, it can be replaced to a new one soon, thereby being sanitary.

### BACKGROUND OF THE INVENTION

1. Field of the Invention

The present invention relates to a biological information processing device which can detect biological information (an electric signal) of the human body by a biological information detection sensor attached to the human body, send the detected biological information, and communicate the detected or sent biological information with an external device.

2. Description of the Related Art

In recent years, in order to respond the demand of medical check-up or the like, a biological information detection sensor which is attached to a site to be detected of a user and detects biological information has been developed, and downsizing of the biological information detection sensor is developing in combination with downsizing of an electronic device (see Patent Document 1: Japanese Published Patent Application No. 2003-275183).

### SUMMARY OF THE INVENTION

It is unsanitary that the biological information detection sensor is constantly in contact with the human body, and it might have an adverse effect on the skin of the human body.

It is an object of the present invention to provide a biological information detection sensor device which can be easily replaced.

The present invention relates to a biological information detection sensor device having an adhesive tape including an adhesive surface which is provided with an adhesive material and a surface which is not provided with the adhesive material; a biological information detection sensor attached on an adhesive surface side of the adhesive tape, which includes a biological information detection portion, a memory portion, an arithmetic processing circuit portion, and a communication circuit portion; and an antenna which is connected to the biological information detection sensor and is pulled from the adhesive surface of the adhesive tape to the surface thereof through a groove provided in the adhesive tape, where biological information is stored in the memory portion.

The present invention also relates to a biological information detection sensor device having an adhesive tape including an adhesive surface which is provided with an adhesive material and a surface which is not provided with the adhesive material; a biological information detection sensor attached on an adhesive surface side of the adhesive tape, which includes a biological information detection portion, and a memory portion, an arithmetic processing circuit portion, and a communication circuit portion, each of which is formed of a thin film transistor in which a channel region is formed of a semiconductor layer which is formed over an insulating surface and is separated to have an island-shape; and an antenna which is connected to the biological information detection sensor and is pulled from the adhesive surface of the adhesive tape to the surface thereof through a groove provided in the adhesive tape, where biological information is stored in the memory portion.

In the present invention, a groove is provided on a surface of the adhesive tape, an adhesive material is provided on a bottom surface of the groove, and the antenna is fixed by the adhesive material.

In the present invention, a groove is provided on a surface of the adhesive tape and a protrusion is provided on side surfaces of the groove so that the antenna is fit.

The present invention also relates to a biological information detection sensor device having an adhesive tape including an adhesive surface which is provided with an adhesive material and a surface which is not provided with the adhesive material; a biological information detection sensor formed on an adhesive surface side of the adhesive tape, which includes an ID chip (also referred to as an IC chip, an ID tag, an IC tag, an RFID, or a wireless tag) and a biological information detection portion; and an antenna which is connected to the biological information detection sensor and is pulled from the adhesive surface of the adhesive tape to the surface thereof through a groove provided in the adhesive tape, where biological information is stored in the ID chip.

In the present invention, information stored in the ID chip and information detected by the sensor are sent and received by the antenna.

In the present invention, the antenna is fixed to the surface of the adhesive tape by a fixation mechanism provided on the surface of the adhesive tape. The fixation mechanism is a mechanism in which a groove is provided on the surface of the adhesive tape and an adhesive material is provided on a bottom surface of the groove so that the antenna is attached, or a mechanism in which a groove is provided on the surface of the adhesive tape and a protrusion is provided on side surfaces of the groove so that the antenna is fit.

Since the biological information detection sensor device of the present invention is directly in contact with the skin of the human body, an external side thereof might easily get dirty due to sweat or the like. In addition, a semiconductor layer of a transistor included in the biological information detection sensor device might be contaminated by an alkali metal element contained in sweat, and accordingly, an electric characteristic of the transistor might be deteriorated. However, according to the present invention, even if the biological information detection sensor gets dirty, it can be easily replaced with a new one, thereby being sanitary.

In addition, the biological information detection sensor system of the present invention can be manufactured at low costs; therefore, it is easy to replace it with a new one, and people can constantly put on a sanitary biological information detection sensor.

### BRIEF DESCRIPTION OF DRAWINGS

In the accompanying drawings:

FIGS. 1A and 1B are views each showing a biological information detection sensor of the present invention;

FIGS. 2A and 2B are views each showing a biological information detection sensor of the present invention;

FIG. 3 is a view showing a biological information detection sensor of the present invention;

FIGS. 4A to 4D are views each showing a biological information detection sensor of the present invention;

FIGS. 5A and 5B are views each showing a manufacturing process of an ID chip of the present invention;

FIGS. 6A and 6B are views each showing a manufacturing process of an ID chip of the present invention;

FIGS. 7A and 7B are views each showing a manufacturing process of an ID chip of the present invention;

FIGS. 8A and 8B are views each showing a manufacturing process of an ID chip of the present invention;

FIGS. 9A and 9B are views each showing a manufacturing process of an ID chip of the present invention;

FIGS. 10A and 10B are views each showing a manufacturing process of an ID chip of the present invention;

FIGS. 11A and 11B are views each showing a manufacturing process of an ID chip of the present invention;

FIGS. 12A and 12B are views each showing a manufacturing process of an ID chip of the present invention;

FIG. 13 is a view showing a structure of an ID chip of the present invention;

FIG. 14 is a view showing a structure of an ID chip of the present invention;

FIG. 15 is a view showing a structure of a reader/writer module of the present invention;

FIG. 16 is a perspective view showing a structural example of an ID chip of the present invention;

FIG. 17 is a block diagram for explaining a structure of a sensor portion of the present invention;

FIGS. 18A and 18B are a block diagram and a circuit diagram for explaining a structure of a sensor portion of the present invention, respectively; and

FIG. 19 is a block diagram for explaining a structure of a sensor portion of the present invention.

### DETAILED DESCRIPTION OF THE INVENTION

### Embodiment Mode

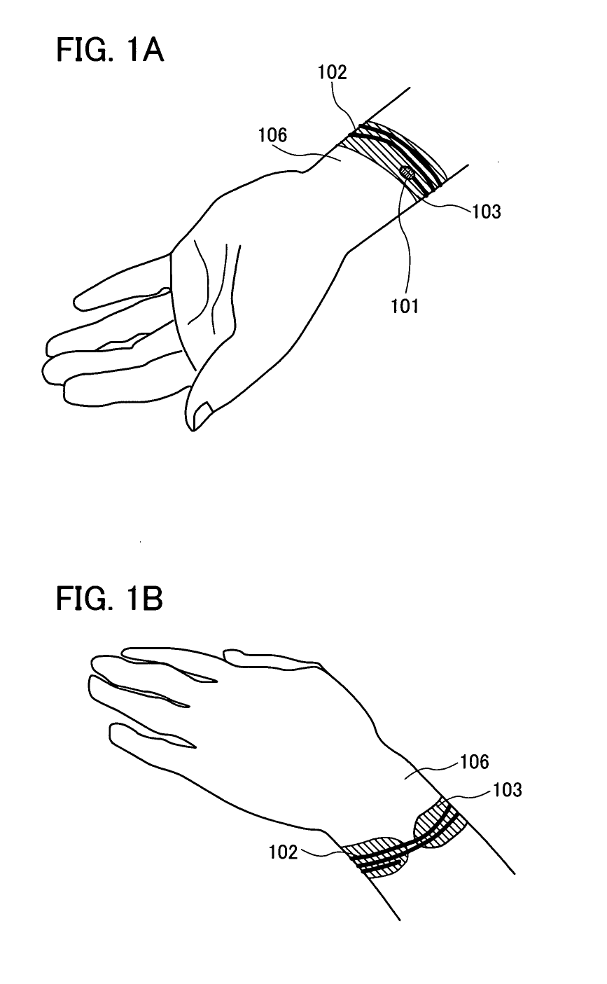

This embodiment mode will be explained with reference to FIGS. 1A and 1B, FIGS. 2A and 2B, and FIG. 3.

A biological information detection sensor 101 is attached to part of a human wrist 106 where the pulse is felt, by an adhesive tape 103. The adhesive tape 103 has an adhesive surface which is provided with an adhesive material and a surface which is not provided with the adhesive material. The biological information detection sensor 101 is attached to the human wrist 106 by the adhesive material provided on the adhesive surface. A humidity sensor, a body temperature sensor, a pulse sensor, and an ID chip in which personal information is stored and the like are built in the biological information detection sensor 101 (see FIGS. 1A and 1B). In this specification, a combination of the biological information detection sensor 101 and the adhesive tape 103 for attaching the biological information detection sensor 101 is referred to as a biological information detection sensor device or a biological information detection sensor system. It is to be noted that the biological information detection sensor 101 is attached to the wrist in FIGS. 1A and 1B; however, a place to which the biological information detection sensor 101 is attached may be changed, such as feet, ears, or a neck, according to need, without being limited to the wrist.

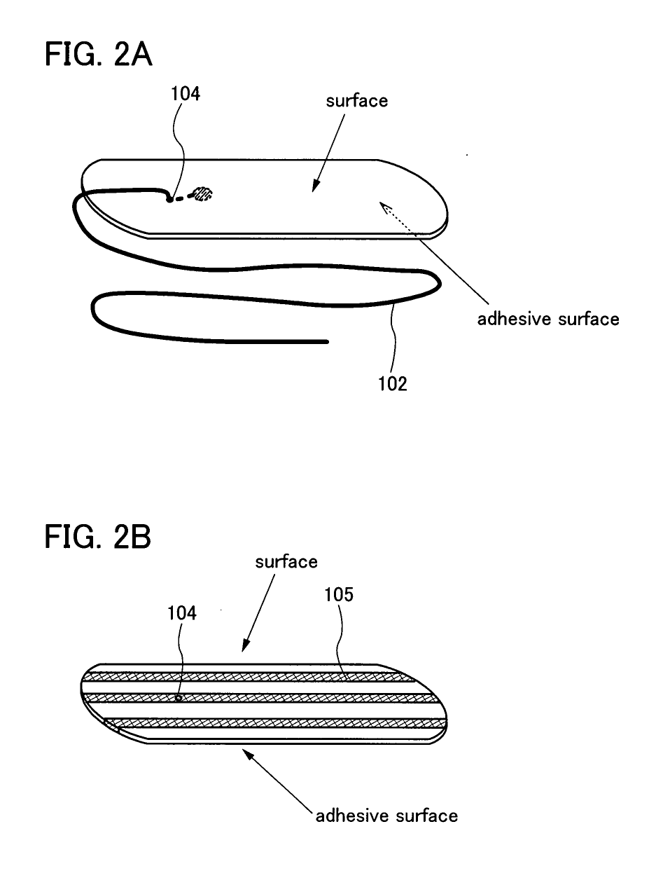

The biological information detection sensor 101 is provided with an antenna 102 for sending or receiving information. The antenna 102 is pulled from the adhesive surface of the adhesive tape 103 to the surface which is not provided with the adhesive material through a groove 104. The surface of the adhesive tape 103 is provided with a fixation mechanism 105 for fixing the antenna 102, and the antenna 102 is fixed to the surface of the adhesive tape 103 after the adhesive tape 103 is fixed to the wrist 106 (see FIGS. 2A and 2B).

For example, the fixation mechanism 105 may have a structure in which a groove is provided on the surface of the adhesive tape 103 and an adhesive material is provided on a bottom surface of the groove so that the antenna 102 is attached. Alternatively, a groove may be provided on the surface of the adhesive tape 103 and a protrusion may be provided on side surfaces of the groove so that the antenna 102 is fit.

If the antenna 102 is too long to be fit into the fixation mechanism on the surface of the adhesive tape 103, the antenna may be attached to the wrist with the use of another adhesive tape.

Since the fixation mechanism for the antenna 102 is provided on the surface of the adhesive tape 103, the antenna 102 does not disturb daily living activities of the user.

Data such as the user's name, sex, blood type, date of birth, previous disease, height, and weight is recorded in advance in the ID chip built in the biological information detection sensor 101.

The pulse sensor built in the biological information detection sensor 101 sends detected pulse data to an external memory through the antenna 102. The sent pulse data is compared with pulse data which has been registered in advance in the external memory to be judged whether or not the pulse is normal.

As for body temperature, similarly to the case of pulse, the body temperature sensor built in the biological information detection sensor 101 sends detected body temperature data to the external memory through the antenna 102. The sent body temperature data is compared with temperature data which has been registered in advance in the external memory to be judged whether or not the body temperature is normal.

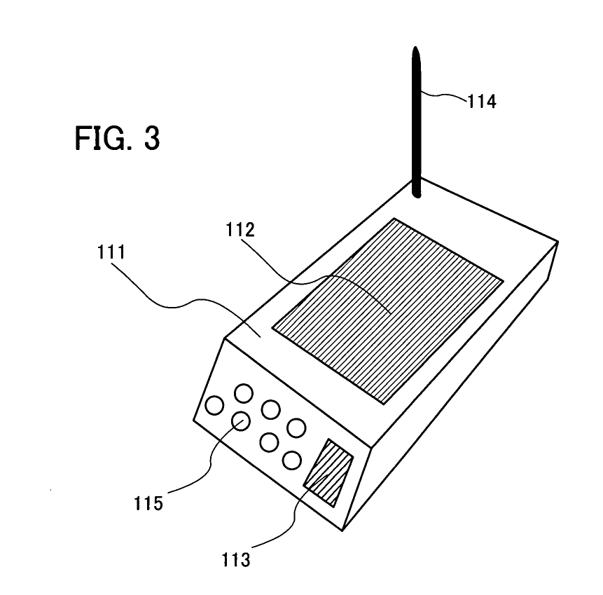

The detected pulse data and body temperature data are sent through the antenna 102, and the data is loaded in a reading (R)/writing (W) machine (hereinafter, referred to as an “R/W machine”) 111 through an antenna 114 of the R/W machine 111. The loaded data is stored in internal memory built in the R/W machine 111. Pulse data, body temperature data, or the like has been registered in advance in the embedded memory of the R/W machine 111 so that the data is compared with the data sent from the biological information detection sensor 101 and is examined (see FIG. 3).

When the data are compared with each other and the sent data is judged to be abnormal, an alarm is displayed on a display portion 112 of the R/W machine 111 to inform abnormality. At this time, the display portion 112 may show how much the value of the abnormal data differs from the normal value.

The R/W machine 111 has another display portion 113 which displays the condition of the R/W machine 111 itself. Operation buttons 115 are used for switching display, stopping the alarm, and turning on and off the power, according to need.

If there are changes of the information recorded in the ID chip built in the biological information detection sensor 101, the R/W machine 111 can rewrite the information. For example, if there are changes of height, weight, previous disease, and the like among the information stored in the ID chip, they can be rewritten by the R/W machine 111.

For rewriting information, data is formed by the operation buttons 115 and the data is sent to the biological information detection sensor 101 with a wireless signal by the antenna 114. The internal information of the ID chip inside the biological information detection sensor 101 is rewritten by the information received through the antenna 102.

Alternatively, without forming the data directly by the R/W machine 111, the data may be formed by another computer or the like, and the data may be sent to the ID chip through the R/W machine 111.

It is preferable that the R/W machine 111 be portable and be placed close to human who puts on the biological information detection sensor 101.

It is to be noted that this embodiment mode can be combined with embodiments, if necessary.

### Embodiment 1

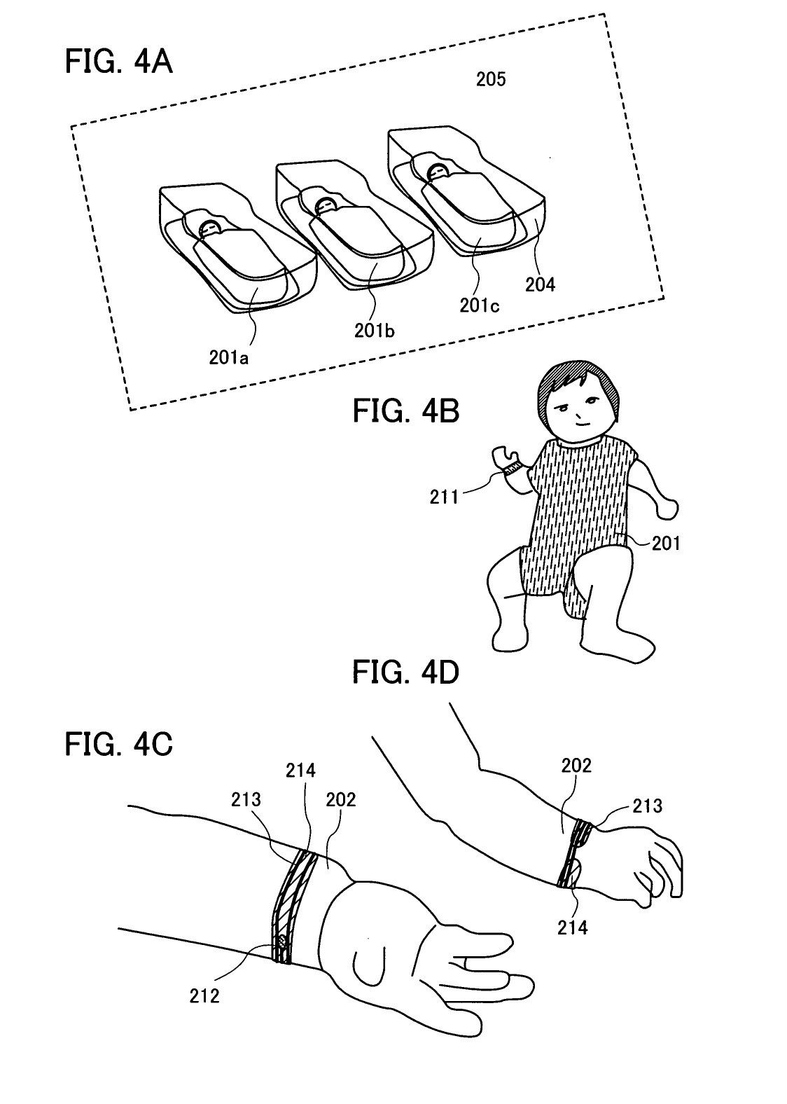

In this embodiment, an example in which a biological information detection sensor of the present invention is applied to infants including newborn babies with reference to FIGS. 4A to 4D.

Newborn babies 201 (201a, 201b, 201c, and so on) lie on beds 204 placed in a nursery room 205 of a hospital (see FIG. 4A). A biological information detection sensor system 211 is attached to a wrist 202 of the newborn baby 201 (see FIG. 4B).

The biological information detection sensor system 211 has the structure described in Embodiment Mode. That is, the biological information detection sensor 212 is attached to the wrist 202 by an adhesive tape 214. The biological information detection sensor 212 is provided with an antenna 213 for sending and receiving. The antenna 213 is pulled from an adhesive surface to a surface through a groove provided in the adhesive tape 214, and is fixed to the surface of the adhesive tape 214 by a fixation mechanism provided on the surface of the adhesive tape 214.

Alternatively, as described in Embodiment Mode, a groove may be provided on the surface of the adhesive tape and an adhesive material may be provided on a bottom surface of the groove so that the antenna is fixed. Alternatively, a protrusion may be provided on side surfaces of the groove so that the antenna is fit.

A humidity sensor, a body temperature sensor, a pulse sensor, and an ID chip in which personal information is recorded and the like are built in the biological information detection sensor 212. The personal information means date of birth, sex, blood type, parents' name, height, weight, and the like. The biological information detection sensor 212 may be put on after storing the personal information in advance in the ID chip. Things that are changed, such as height and weight, may be rewritten in every measurement. In addition, as for things identified after a test, such as blood type, they may be recorded after the biological information detection sensor 212 is put on.

The personal information is stored in the ID chip in the biological information detection sensor 212, so that the newborn baby 201 can be easily identified and baby mix-up by some chance can be prevented. In addition, information such as height, weight, and blood type can be easily known.

Health management of the newborn baby 201 can be practiced by various sensors, such as the humidity sensor, the body temperature sensor, and the pulse sensor, built in the biological information detection sensor 212. For example, body temperature is constantly observed by the body temperature sensor, and an alarm is set to be given when the body temperature is higher than a certain temperature, whereby an immediate response is possible even when something is wrong with his/her body.

Since infants including newborn babies have fast metabolism and sensitive skin, the biological information detection sensor system of this embodiment that can be easily replaced frequently has advantages of being sanitary and easy-to-use.

Moreover, a device sending positional information may be built in the biological information detection sensor 212, if necessary. The device sending positional information which is provided in the biological information detection sensor 212 and a detector for detecting the device make it possible to identify where in the hospital the newborn baby is, such as the nursery room 205, mother's room, or an outside thereof.

It is to be noted that this embodiment can be combined with Embodiment Mode and other embodiments, if necessary.

### Embodiment 2

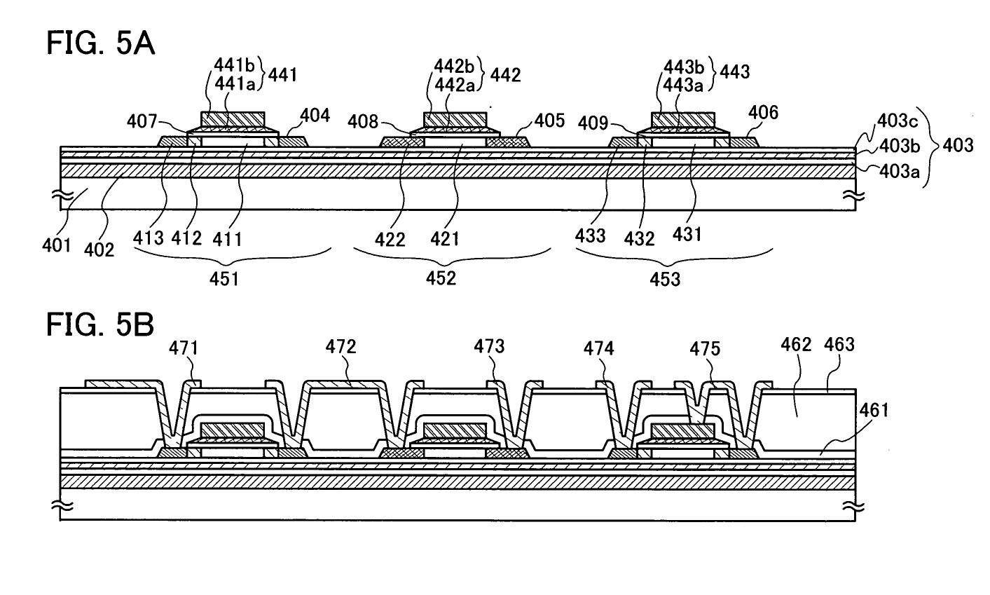

In this embodiment, an example in which an ID chip (also referred to as an IC tag or an IC chip) is manufactured by the present invention will be described with reference to FIGS. 5A and 5B, FIGS. 6A and 6B, FIGS. 7A and 7B, and FIGS. 8A and 8B.

In this embodiment, a TFT that is insulated and isolated is exemplified as a semiconductor element; however, a semiconductor element used for an integrated circuit is not limited thereto, and all circuit elements can be used. For example, a memory element, a diode, a photoelectric conversion element, a resistance element, a coil, a capacitor element, an inductor, and the like are typically given, besides a TFT.

First, a separation layer 402 is formed over a substrate (a first substrate) 401 having heat resistance by a sputtering method. For example, as the first substrate 401, a glass substrate such as barium borosilicate glass or aluminoborosilicate glass, a quartz substrate, a ceramic substrate, or the like can be used. Alternatively, a metal substrate including a stainless steel substrate or a semiconductor substrate over which an insulating film is formed may be used. A substrate formed of a synthetic resin having flexibility, such as plastic, generally tends to have lower allowable temperature limit than the above-described substrates; however, the substrate can be used as long as it can resist a processing temperature in manufacturing steps.

As the separation layer 402, a layer containing silicon such as amorphous silicon, polycrystalline silicon, single-crystal silicon, or microcrystalline silicon (including semi-amorphous silicon) as its main component can be used. The separation layer 402 can be formed by a sputtering method, a low-pressure CVD method, a plasma CVD method, or the like. In this embodiment, amorphous silicon formed with a thickness of approximately 50 nm by a low-pressure CVD method is used as the separation layer 402. Further, the separation layer 402 is not limited to silicon, and a material which can be selectively removed by etching may be used. It is desirable that the thickness of the separation layer 402 be 50 to 60 nm. If semi-amorphous silicon is used, the thickness thereof may be 30 to 50 nm.

It is to be noted that a semi-amorphous semiconductor typified by semi-amorphous silicon is a film containing a semiconductor having an intermediate structure between an amorphous semiconductor and a semiconductor having a crystalline structure (including single crystal and polycrystalline structures). This semi-amorphous semiconductor has a third state which is stable in terms of free energy, and which is crystalline having a short range order and lattice distortion. The semi-amorphous semiconductor can be formed with a grain diameter of 0.5 to 20 nm to be dispersed in a non single-crystal semiconductor. A Raman spectrum of the semi-amorphous silicon is shifted to a lower wavenumber than 520 cm⁻¹. The diffraction peaks of (111) and (220) which are thought to be derived from a silicon (Si) crystalline lattice are observed by X-ray diffraction. Hydrogen or halogen of at least 1 atomic % or more is contained as a material for terminating dangling bonds. Here, such a semiconductor is referred to as a semi-amorphous semiconductor (SAS) for the sake of convenience. A favorable semi-amorphous semiconductor with increased stability can be obtained by further promotion of the lattice distortion by means of adding a rare gas element such as helium, argon, krypton, or neon.

Semi-amorphous silicon can be obtained by glow discharge decomposition of a gas containing silicon. As the typical gas containing silicon, SiH₄ is given, and besides, Si₂H₆, SiH₂Cl₂, SiHCl₃, SiCl₄, SiF₄, or the like can be used. Semi-amorphous silicon can be easily formed with the use of the gas containing silicon diluted with hydrogen or a gas in which one or more of rare gas elements selected from helium, argon, krypton, and neon is/are added to hydrogen. It is preferable to dilute the gas containing silicon at a dilution rate in the range of 2 to 1000 times.

Subsequently, a base film 403 is formed over the separation layer 402. The base film 403 is provided to prevent an alkali metal such as Na or an alkaline earth metal contained in the first substrate 401 from diffusing into a semiconductor film and having an adverse effect on an electric characteristic of a semiconductor element such as a TFT. In addition, the base film 403 also has a role of protecting the semiconductor element in a later step of separating the semiconductor element. The base film 403 may be a single layer or a stacked layer of a plurality of insulating films. Consequently, the base film 403 is formed using an insulating film such as silicon oxide, silicon nitride, silicon oxide containing nitrogen, silicon nitride containing oxygen, or the like, which can suppress diffusion of an alkali metal or an alkaline earth metal into a semiconductor film.

In this embodiment, a silicon oxide film containing nitrogen with a thickness of 100 nm as a lower base film 403a, a silicon nitride film containing oxygen with a thickness of 50 nm as a middle base film 403b, and a silicon oxide film containing nitrogen with a thickness of 100 nm as an upper base film 403c are stacked in this order to form the base film 403. However, a material and a thickness of each film, and the number of stacked layers are not limited thereto. For example, instead of the silicon oxide film containing nitrogen, which is the lower base film 403a, a siloxane-based resin may be formed with a thickness of 0.5 to 3 μm by a spin coating method, a slit coating method, a droplet discharging method, or the like. Instead of the silicon nitride film containing oxygen, which is the middle base film 403b, a silicon nitride film (Si₃N₄ or the like) may be used. Instead of the silicon oxide film containing nitrogen, which is the upper base film 403c, a silicon oxide film may be used. It is desirable that the thickness of each film be 0.05 to 3 μm, and the thickness can be freely selected from this range.

Alternatively, the lower base film 403a of the base film 403, which is closest to the separation layer 402, may be formed of a silicon oxide film containing nitrogen or a silicon oxide film, the middle base film 403b may be formed of a siloxane-based resin, and the upper base film 403c may be formed of a silicon oxide film.

Here, the silicon oxide film can be formed using a mixed gas of SiH₄ and O₂, TEOS (tetraethoxysilane) and O₂, or the like, by a method such as thermal CVD, plasma CVD, normal pressure CVD, or bias ECRCVD. Also, the silicon nitride film can be formed typically using a mixed gas of SiH₄ and NH₃, by plasma CVD. Furthermore, the silicon oxide film containing nitrogen (composition ratio O>N) and the silicon nitride film containing oxygen (composition ratio N>O) can be formed typically using a mixed gas of SiH₄ and N₂O, by plasma CVD.

Subsequently, a semiconductor film is formed over the base film 403. It is preferable that, after forming the base film 403, the semiconductor film be formed without exposure to the air. The thickness of the semiconductor film is 20 to 200 nm (desirably, 40 to 170 nm, preferably, 50 to 150 nm). Further, the semiconductor film may be an amorphous semiconductor, a semi-amorphous semiconductor, or a polycrystalline semiconductor. Not only silicon but also silicon germanium can be used as the semiconductor. In the case of using silicon germanium, a concentration of the germanium is preferably about 0.01 to 4.5 atomic %.

The amorphous semiconductor can be obtained by glow discharge decomposition of a gas containing silicon. As the typical gas containing silicon, SiH₄ and Si₂H₆ are given. Those gases containing silicon may be used by dilution with hydrogen, or hydrogen and helium.

As described above, the semi-amorphous semiconductor can be obtained by glow discharge decomposition of the gas containing silicon. However, a carbide gas such as CH₄ or C₂H₆, a germanium gas such as GeH₄ or GeF₄, or F₂ may be mixed into the gas containing silicon to adjust the width of the energy band to 1.5 to 2.4 eV or 0.9 to 1.1 eV.

For example, in the case of using a gas in which H₂ is added to SiH₄ or the gas in which F₂ is added to SiH₄, the subthreshold coefficient (S value) of the TFT can be less than or equal to 0.35 V/dec, typically, 0.25 to 0.09 V/dec, and the mobility can be 10 cm²/Vs when the TFT is manufactured using the formed semi-amorphous semiconductor. When 19-stage ring oscillator is formed of a TFT using the above-described semi-amorphous semiconductor, for example, a property in which a repetition rate is greater than or equal to 1 MHz, preferably, greater than or equal to 100 MHz, with power supply voltage of 3 to 5V, can be obtained. In addition, in the power supply voltage of 3 to 5V, delay time per one stage of an inverter can be 26 ns, preferably, less than or equal to 0.26 ns.

Then, the semiconductor film is crystallized using a laser. Alternatively, a crystallization method in which a catalyst element is used and a laser crystallization method in which a laser is used may be combined.

In the case of performing laser crystallization, before performing the laser crystallization, heat treatment at 500° C. for 1 hour may be performed to the semiconductor film in order to increase resistance of the semiconductor film to the laser.

For the laser crystallization, a continuous wave laser (a CW laser) can be used, or alternatively, a pulse oscillation laser at a repetition rate of greater than or equal to 10 MHz, preferably, greater than or equal to 80 MHz can be used as a pseudo CW laser.

Specifically, the following can be given as the continuous wave laser: an Ar laser, a Kr laser, a CO₂ laser, a YAG laser, a YVO₄ laser, a forsterite (Mg₂SiO₄) laser, a YLF laser, a YAlO₃ laser, a GdVO₄ laser, a Y₂O₃ laser, an alexandrite laser, a Ti:sapphire laser, a helium cadmium laser, and a laser of which a medium is a polycrystalline (ceramic) YAG, Y₂O₃, YVO₄, YAlO₃, or GdVO₄ doped with one or more of Nd, Yb, Cr, Ti, Ho, Er, Tm, and Ta as a dopant.

As the pseudo CW laser, the following can be used as long as pulse oscillation with a repetition rate of greater than or equal to 10 MHz, preferably, greater than or equal to 80 MHz is possible: the pulse oscillation laser such as an Ar laser, a Kr laser, an excimer laser, a CO₂ laser, a YAG laser, a Y₂O₃ laser, a YVO₄ laser, a forsterite (Mg₂SiO₄) laser, a YLF laser, a YAlO₃ laser, a GdVO₄ laser, an alexandrite laser, a Ti:sapphire laser, a copper vapor laser, a gold vapor laser, or a laser of which a medium is a polycrystalline (ceramic) YAG, Y₂O₃, YVO₄, YAlO₃, or GdVO₄ doped with one or more of Nd, Yb, Cr, Ti, Ho, Er, Tm, and Ta as a dopant.

Such a pulse oscillation laser eventually shows an effect equivalent to that of a continuous wave laser when the repetition rate is increased.

For example, in the case of using a solid-state laser capable of continuous oscillation, a crystal with a large grain diameter can be obtained by irradiation with laser light of the second to fourth harmonics. Typically, it is desirable to use the second harmonic (532 nm) or the third harmonic (355 nm) of the YAG laser (fundamental wave of 1064 nm). For example, laser light emitted from a continuous wave YAG laser is converted into a high harmonic by a nonlinear optical element, and emitted to the semiconductor film. An energy density may be approximately 0.01 to 100 MW/cm² (preferably 0.1 to 10 MW/cm²). Then, irradiation is carried out at a scanning speed of approximately 10 to 2000 cm/s.

It is to be noted that a laser of which a medium is a single crystal YAG, YVO₄, forsterite (Mg₂SiO₄), YAlO₃, or GdVO₄ doped with one or more of Nd, Yb, Cr, Ti, Ho, Er, Tm, and Ta as a dopant, or a polycrystalline (ceramic) YAG, Y₂O₃, YVO₄, YAlO₃, or GdVO₄ doped with one or more of Nd, Yb, Cr, Ti, Ho, Er, Tm, and Ta as a dopant; an Ar laser; a Kr laser; or a Ti:sapphire laser is capable of continuous oscillation, and is also capable of pulse oscillation by a Q switch operation, mode locking, or the like. When a laser beam is oscillated at a repetition rate of greater than or equal to 10 MHz, the semiconductor film is irradiated with a subsequent pulse while the semiconductor film is melted by a preceding laser and then solidified. Consequently, since a solid-liquid interface in the semiconductor film can be moved continuously unlike in the case of using a pulse laser with a low repetition rate, crystal grains that continuously grow towards a scanning direction can be obtained.

When a ceramic (a poly crystal) is used for a medium, the medium can be formed into a free shape in a short amount of time and at low costs. In the case of using a single crystal, a column-shaped medium with several mm in diameter and several ten mm in length is usually used; however, a larger medium can be formed in the case of using ceramic.

Since a concentration of a dopant such as Nd or Yb in the medium which directly contributes to light emission cannot be changed significantly in either a single crystal or a poly crystal, improvement in laser output by increase of the concentration is limited to a certain extent. However, in the case of ceramic, there is a possibility that output can be drastically improved since the size of the medium can be significantly increased in comparison with a single crystal.

Furthermore, in the case of ceramic, a medium having a parallelepiped shape or a rectangular parallelepiped shape can be easily formed. When a medium having such a shape is used and oscillation light goes in zigzag in the medium, an oscillation light path can be longer. Accordingly, amplification is increased and oscillation with high output is possible. Since a laser beam emitted from the medium having such a shape has a cross section of a quadrangular shape when being emitted, a linear beam can be easily shaped in comparison with the case of a circular beam. The laser beam emitted in such a manner is shaped with the use of an optical system; accordingly, a linear beam having a short side of less than or equal to 1 mm and a long side of several mm to several m can be easily obtained. In addition, by uniform irradiation of the medium with excited light, the linear beam has a uniform energy distribution in a long side direction.

By irradiation of the semiconductor film with this linear beam, an entire surface of the semiconductor film can be annealed more uniformly. In the case where uniform annealing is necessary from one edge to the other edge of the linear beam, slits may be provided for both edges so as to shield a portion where energy is attenuated from light.

By irradiation of the semiconductor film with the above-described laser light, a crystalline semiconductor film with further increased crystallinity is formed.

Subsequently, island-shaped semiconductor films 404, 405, and 406 are formed using the crystalline semiconductor film. Each of the island-shaped semiconductor films 404, 405, and 406 becomes an active layer of a TFT.

Next, an impurity for controlling a threshold value is introduced into the island-shaped semiconductor film. In this embodiment, boron (B) is introduced into the island-shaped semiconductor film by doping of diborane (B₂H₆).

Then, an insulating film is formed so as to cover the island-shaped semiconductor films 404, 405, and 406. For example, silicon oxide, silicon nitride, silicon oxide containing nitrogen, or the like can be used for the insulating film. As a formation method, a plasma CVD method, a sputtering method, or the like can be used.

Next, a conductive film is formed over the insulating film, and then, gate electrodes 441, 442, and 443 are formed using the conductive film.

Each of the gate electrodes 441, 442, and 443 is formed to have a structure including a single layer or two or more stacked layers of a conductive film. In the case where two or more layers of the conductive film are stacked, an element selected from tantalum (Ta), tungsten (W), titanium (Ti), molybdenum (Mo), and aluminum (Al), an alloy material or compound material containing the above-described element as its main component may be stacked to form the gate electrodes 441, 442, and 443. Alternatively, the gate electrode may be formed using a semiconductor film typified by a polycrystalline silicon film doped with an impurity element such as phosphorus (P).

In this embodiment, the gate electrodes 441, 442, and 443 are formed in the following manner. First, for example, a tantalum nitride (TaN) film is formed with a thickness of 10 to 50 nm, for example, 30 nm as a first conductive film. Then, for example, a tungsten (W) film is formed with a thickness of 200 to 400 nm, for example, 370 nm as a second conductive film over the first conductive film, so that a stacked film of the first conductive film and the second conductive film is formed.

Subsequently, the second conductive film is etched by anisotropic etching to form upper gate electrodes 441b, 442b, and 443b. Then, the first conductive film is etched by isotropic etching to form lower gate electrodes 441a, 442a, and 443a. Accordingly, the gate electrodes 441, 442, and 443 are formed.

The gate electrodes 441, 442, and 443 may be used as part of a gate wiring. Alternatively, another gate wiring may be formed to connect the gate electrodes 441, 442, and 443 thereto.

Then, an impurity imparting one conductivity (n-type or p-type conductivity) is added to each of the island-shaped semiconductor films 404, 405, and 406 with the use of the gate electrodes 441, 442, and 443, or a resist as a mask, so that a source region, a drain region, furthermore a low concentration impurity region, and the like is formed.

First, phosphorus (P) is introduced into the island-shaped semiconductor film with the use of phosphine (PH₃) with acceleration voltage of 60 to 120 keV and a dose amount of 1×10¹³ to 1×10¹⁵ cm⁻². When introducing the impurity, a channel formation region 411 of an n-channel TFT 451 and a channel formation region 431 of an n-channel TFT 453 are formed.

In addition, in order to manufacture a p-channel TFT 452, boron (B) is introduced into the island-shaped semiconductor film with the use of diborane (B₂H₆) with applied voltage of 60 to 100 keV, for example, 80 keV, and a dose amount of 1×10¹³ to 5×10¹⁵ cm⁻², for example, 3×10¹⁵ cm⁻². Accordingly, a source region or drain region 422 of the p-channel TFT 452 is formed, and furthermore, a channel formation region 421 is formed when introducing the impurity.

Next, gate insulating films 407, 408, and 409 are formed using an insulating film.

After forming the gate insulating films 407, 408, and 409, phosphorus (P) is introduced into the island-shaped semiconductor films that become the n-channel TFTs 451 and 453 with the use of phosphine (PH₃) with applied voltage of 40 to 80 keV, for example, 50 keV, and a dose amount of 1.0×10¹⁵ to 2.5×10¹⁶ cm⁻², for example, 3.0×10¹⁵ cm⁻². Accordingly, a low concentration impurity region 412 and a source or drain region 413 of the n-channel TFT 451, and a low concentration impurity region 432 and a source or drain region 433 of the n-channel TFT 453 are formed.

In this embodiment, phosphorus (P) is contained in each of the source or drain region 413 of the n-channel TFT 451 and the source or drain region 433 of the n-channel TFT 453 at a concentration of 1×10¹⁹ to 5×10²¹ cm⁻³. In addition, phosphorus (P) is contained in each of the low concentration impurity region 412 of the n-channel TFT 451 and the low concentration impurity region 432 of the n-channel TFT 453 at a concentration of 1×10¹⁸ to 5×10¹⁹ cm³. Furthermore, boron (B) is contained in the source or drain region 422 of the p-channel TFT 452 at a concentration of 1×10¹⁹ to 5×10²¹ cm⁻³.

Accordingly, the n-channel TFT 451, the p-channel TFT 452, and the n-channel TFT 453 are formed (see FIG. 5A). It is to be noted that, in this embodiment, each of the TFTs 451, 452, and 453 has a top gate structure; however, a bottom gate structure (an inversely staggered structure) may also be employed.

The n-channel TFT 451 has the island-shaped semiconductor film 404, the gate insulating film 407, and the gate electrode 441 including the lower gate electrode 441a and the upper gate electrode 441b, over the upper base film 403c. In the island-shaped semiconductor film 404, the channel formation region 411, the low concentration impurity region 412, and the source or drain region 413 are formed.

The p-channel TFT 452 has the island-shaped semiconductor film 405, the gate insulating film 408, and the gate electrode 442 including the lower gate electrode 442a and the upper gate electrode 442b, over the upper base film 403c. In the island-shaped semiconductor film 405, the channel formation region 421 and the source or drain region 422 are formed.

The n-channel TFT 453 has the island-shaped semiconductor film 406, the gate insulating film 409, and the gate electrode 443 including the lower gate electrode 443a and the upper gate electrode 443b, over the upper base film 403c. In the island-shaped semiconductor film 406, the channel formation region 431, the low concentration impurity region 432, and the source or drain region 433 are formed.

Furthermore, after that, a passivation film 461 for protecting the TFTs 451, 452, and 453 may be formed. As the passivation film 461, it is desirable to use silicon nitride, silicon oxide containing nitrogen, aluminum nitride, aluminum oxide, silicon oxide, or the like which can prevent an alkali metal or an alkaline earth metal from entering the TFTs 451, 452, and 453. Specifically, a silicon oxide film containing nitrogen with a thickness of approximately 600 nm can be used as the passivation film 461, for example. In this case, hydrogenation treatment step may be performed after forming the silicon oxide film containing nitrogen. With the use of the above-described structure, the TFTs 451, 452, and 453 are covered with the base film 403 and the passivation film 461; therefore, an alkali metal such as Na or an alkaline earth metal can be prevented from diffusing into the semiconductor film used for a semiconductor element and having an adverse effect on an electric characteristic of the semiconductor element.

Subsequently, a first interlayer insulating film 462 is formed so as to cover the TFTs 451, 452, and 453 and the passivation film 461. A heat resistant organic resin such as polyimide, acrylic, or polyamide can be used for the first interlayer insulating film 462. Besides the above-described organic resin, a low-dielectric material (a low-k material), a resin including a Si—O—Si bond formed using a siloxane-based material as a starting material (hereinafter, referred to as a siloxane-based resin), or the like can be used.

Siloxane has a skeleton structure formed by a bond of silicon (Si) and oxygen (O) and has an organic group containing at least hydrogen (for example, an alkyl group or an aryl group) as a substituent. Alternatively, as the substituent, a fluoro group may be used. Further alternatively, as the substituent, an organic group containing at least hydrogen and a fluoro group may be used.

The first interlayer insulating film 462 can be formed by spin coating, dipping, spray coating, droplet discharging (ink jetting, screen printing, offset printing, or the like), a doctor knife, a roll coater, a curtain coater, a knife coater, or the like, depending on the material thereof. Alternatively, the first interlayer insulating film 462 can be formed using an inorganic material such as silicon oxide, silicon nitride, silicon oxynitride, PSG (phosphosilicate glass), BPSG (boron phosphosilicate glass), an alumina film, or the like. Further, these insulating films may be stacked to form the first interlayer insulating film 462.

Furthermore, in this embodiment, a second interlayer insulating film 463 is formed over the first interlayer insulating film 462. As the second interlayer insulating film 463, a film containing carbon such as DLC (diamond like carbon) or carbon nitride (CN), a silicon oxide film, a silicon nitride film, a silicon oxide film containing nitrogen, or the like can be used. As a formation method, a plasma CVD method, an atmospheric pressure plasma method, or the like can be used. Alternatively, a photosensitive or nonphotosensitive organic material such as polyimide, acrylic, polyamide, resist, or benzocyclobutene; a siloxane-based resin; or the like may be used.

Filler may be mixed into the first interlayer insulating film 462 or the second interlayer insulating film 463 in order to prevent the first interlayer insulating film 462 or the second interlayer insulating film 463 from being peeled off or cracked due to stress generated by a difference in coefficient of thermal expansion between the first interlayer insulating film 462 or the second interlayer insulating film 463 and a conductive material for forming a wiring that is formed later, or the like.

Next, contact holes are formed in the first interlayer insulating film 462 and the second interlayer insulating film 463 to form electrodes or wirings 471, 472, 473, 474, and 475 connected to the TFTs 451, 452, and 453. In this embodiment, the electrode and the wiring are formed together; however, the electrode and the wiring may be formed separately and electrically connected to each other. Although a mixed gas of CHF₃ and He is used for etching when forming the contact holes, a gas is not limited thereto. In this embodiment, each of the electrodes or wirings 471, 472, 473, 474, and 475 is formed of a five-layered structure of Ti, TiN, Al—Si, Ti, and TiN by a sputtering method.

When silicon (Si) is mixed into an aluminum (Al) film, a hillock can be prevented from generating in resist baking when the wiring is formed. Instead of Si, copper (Cu) of approximately 0.5% may be mixed. In addition, when an Al—Si layer is interposed between titanium (Ti) and titanium nitride (TiN), suppressing hillock development is further improved. Further, it is desirable to use the above-described hard mask formed of silicon oxide containing nitrogen, or the like in etching. It is to be noted that a material or a formation method of the wiring are not limited to these, and the above-described material used for the gate electrode may also be used.

Alternatively, the electrodes or wirings 471, 472, 473, 474, and 475 may be formed of an aluminum alloy film containing carbon and at least one kind of element selected from nickel, cobalt, and iron. Such an aluminum alloy film can prevent interdiffusion of silicon and aluminum even if the electrodes or wirings 471, 472, 473, 474, and 475 are in contact with silicon. Also, with such an aluminum alloy film, oxidation-reduction reaction does not occur even if the aluminum alloy film is in contact with a transparent conductive film, for example, an ITO (indium tin oxide) film, and thus, they can be in direct contact with each other. Furthermore, such an aluminum alloy film has low resistivity and is excellent in heat resistance, and therefore, it is useful as a wiring material.

It is to be noted that the electrode or wiring 471 and the electrode or wiring 472 are connected to the source or drain region 413 of the n-channel TFT 451. The electrode or wiring 472 and the electrode or wiring 473 are connected to the source or drain region 422 of the p-channel TFT 452. The electrode or wiring 474 and the electrode or wiring 475 are connected to the source or drain region 433 of the n-channel TFT 453. Furthermore, the electrode or wiring 475 is also connected to the gate electrode 443 of the n-channel TFT 453. The n-channel TFT 453 can be used as a memory element of a random ROM (see FIG. 5B).

Subsequently, a third interlayer insulating film 464 is formed over the second interlayer insulating film 463 so as to cover the electrodes or wirings 471, 472, 473, 474, and 475. The third interlayer insulating film 464 is formed so as to have an opening at a position where part of the electrode or wiring 471 is exposed. Further, the third interlayer insulating film 464 can be formed using a material similar to that of the first interlayer insulating film 462.

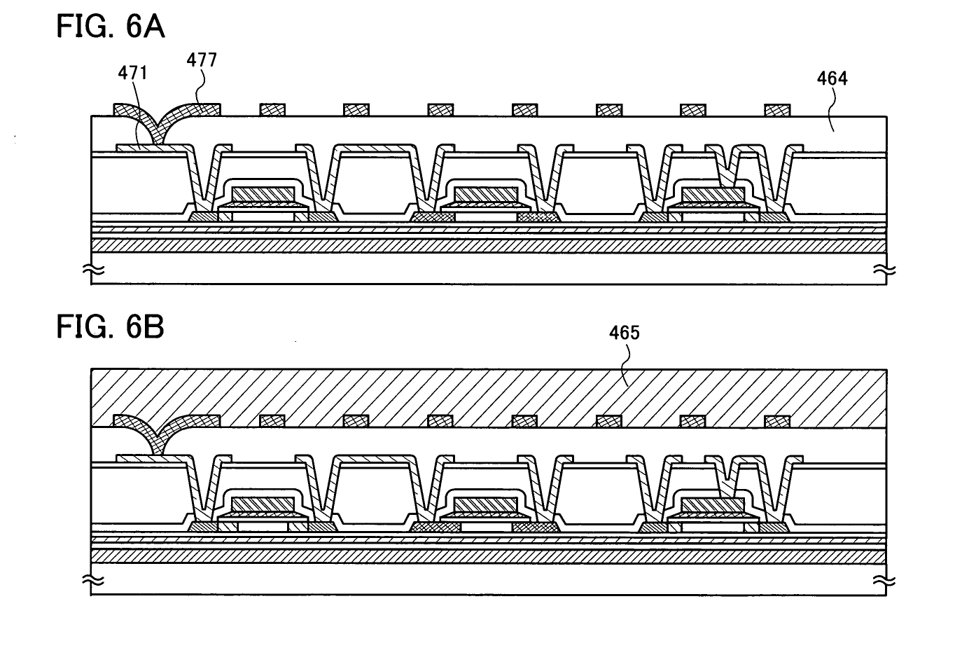

Next, an antenna 477 is formed over the third interlayer insulating film 464 (see FIG. 6A). The antenna 477 can be formed using a conductive material including one or more of metals such as Ag, Au, Cu, Pd, Cr, Mo, Ti, Ta, W, Al, Fe, Co, Zn, Sn, and Ni, and metal compounds thereof. The antenna 477 is connected to the electrode or wiring 471. It is to be noted that, in FIG. 6A, the antenna 477 is directly connected to the electrode or wiring 471; however, a structure of the ID chip of the present invention is not limited thereto. For example, the antenna 477 and the electrode or wiring 471 may be electrically connected to each other with the use of a wiring which is separately formed.

The antenna 477 can be formed by a printing method, a photolithography method, an evaporation method, a droplet discharging method, or the like. In this embodiment, the antenna 477 is formed of a single conductive film; however, the antenna 477 in which a plurality of conductive films is stacked can also be formed. For example, the antenna 477 may be formed in such a manner that a wiring formed of Ni or the like is coated with Cu by electroless plating.

It is to be noted that a droplet discharging method means a method in which droplets containing predetermined composition are discharged from a pore to form a predetermined pattern, and an ink jetting method is included in the category. Also, a screen printing method, an offset printing method, or the like is included in a printing method. The use of a printing method and a droplet discharging method makes it possible to form the antenna 477 without using a mask for exposure. In addition, when a droplet discharging method and a printing method are used, waste of material due to removal by etching does not occur, unlike a photolithography method. Moreover, an expensive mask for exposure is not necessary to be used; therefore, costs spent for manufacturing the ID chip can be reduced.

In the case of using a droplet discharging method or various printing methods, for example, a conductive particle in which Cu is coated with Ag, or the like can also be used. Further, in the case of forming the antenna 477 by a droplet discharging method, it is desirable that treatment for increasing adhesiveness of the antenna 477 be performed to a surface of the third interlayer insulating film 464.

As the method which can increase the adhesiveness, for example, the following can be specifically given: a method in which a metal or metal compound which can increase adhesiveness of a conductive film or an insulating film by catalysis is attached to the surface of the third interlayer insulating film 464, a method in which an organic insulating film, a metal, and a metal compound having high adhesiveness with a conductive film or insulating film to be formed are attached to the surface of the third interlayer insulating film 464, a method in which plasma treatment is performed to the surface of the third interlayer insulating film 464 under atmospheric pressure or reduced pressure to perform surface modification, or the like. As the above-described metal having high adhesiveness with the conductive film or insulating film, Sc, Ti, V, Cr, Mn, Fe, Co, Ni, Cu, Zn, and the like which are 3d transition elements can be given, besides titanium or titanium oxide. As the metal compound, oxide, nitride, and oxynitride of the above-described metal, or the like can be given. As the above-described organic insulating film, for example, polyimide, a siloxane-based resin, or the like can be given.

In the case where the metal or metal compound to be attached to the third interlayer insulating film 464 has conductivity, sheet resistance thereof is controlled so that normal performance of the antenna is not disturbed. Specifically, it is preferable that an average thickness of the metal or metal compound having conductivity be controlled so as to be, for example, 1 to 10 nm, or part or all of the metal or metal compound be insulated by oxidation. Alternatively, the attached metal or metal compound in a region except for a region where the adhesiveness is desired to be increased may be selectively removed by etching. Alternatively, the metal or metal compound may be selectively attached to only a specific region by a droplet discharging method, a printing method, a sol-gel method, or the like without attaching the metal or metal compound on an entire surface of a substrate in advance. It is to be noted that the metal or metal compound is not necessarily in a completely continuous film form and may be dispersed to some extent on the surface of the third interlayer insulating film 464.

Then, as shown in FIG. 6B, after forming the antenna 477, a protective layer 465 is formed over the third interlayer insulating film 464 so as to cover the antenna 477. The protective layer 465 is formed using a material which can protect the antenna 477 when the separation layer 402 is removed by etching later. For example, the protective layer 465 can be formed by application of an epoxy-based resin, an acrylate-based resin, or a resin containing silicon that is soluble in water or alcohols over the entire surface.

In this embodiment, the protective layer 465 is formed in the following manner: a water-soluble resin (manufactured by Toagosei Co., Ltd.: VL-WSHL10) is applied so as to have a thickness of 30 μm by a spin coating method, is exposed to light for 2 minutes for temporary curing, and then, its back surface is exposed to UV light for 2.5 minutes and a front surface is exposed to the UV light for 10 minutes, 12.5 minutes in total, so that the resin is fully cured. Further, in the case of stacking a plurality of organic resins, the stacked organic resins might be partly melted depending on a solvent to be used, during application or baking, or the adhesiveness might become too high. Therefore, in the case of forming both the third interlayer insulating film 464 and the protective layer 465 using an organic resin that is soluble in the same solvent, it is preferable to form an inorganic insulating film (a silicon nitride film, a silicon nitride film containing oxygen, a silicon oxide film containing nitrogen, an aluminum nitride film, an aluminum nitride film containing oxygen, or an aluminum oxide film containing nitrogen) so as to cover the third interlayer insulating film 464 for smoothly removing the protective layer 465 in a later step.

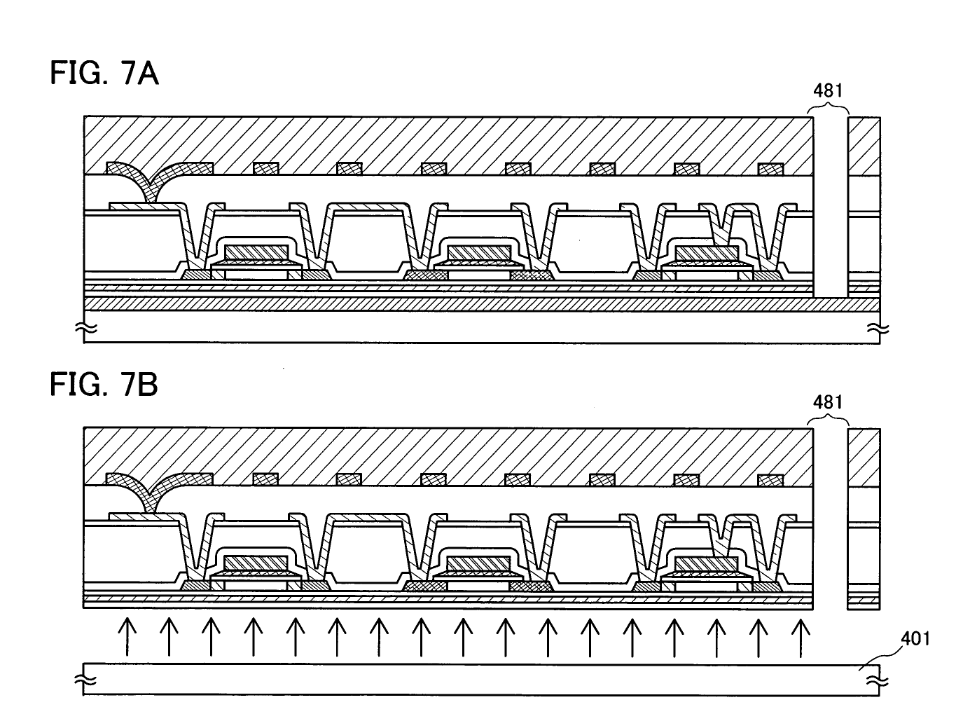

Subsequently, as shown in FIG. 7A, an opening 481 is formed in order to separate the ID chips. The opening 481 is formed to the extent that the separation layer 402 is exposed. The opening 481 can be formed by dicing, scribing, or the like. Further, in the case where the ID chips formed over the first substrate 401 are not necessary to be separated, the opening 481 is not necessarily formed.

Next, as shown in FIG. 7B, the separation layer 402 is removed by etching. In this embodiment, halogen fluoride is used as an etching gas and the gas is introduced from the opening 481. In this embodiment, for example, the etching is performed using ClF₃ (chlorine trifluoride) at a temperature of 350° C. with a flow rate of 300 sccm and air pressure of 798 Pascal (798 Pa) for 3 hours. Alternatively, a gas in which nitrogen is mixed into a ClF₃ gas may be used. When halogen fluoride such as ClF₃ is used, the separation layer 402 is selectively etched and the first substrate 401 can be separated from the TFTs 451, 452, and 453. Further, the halogen fluoride may be either a gas or a liquid.

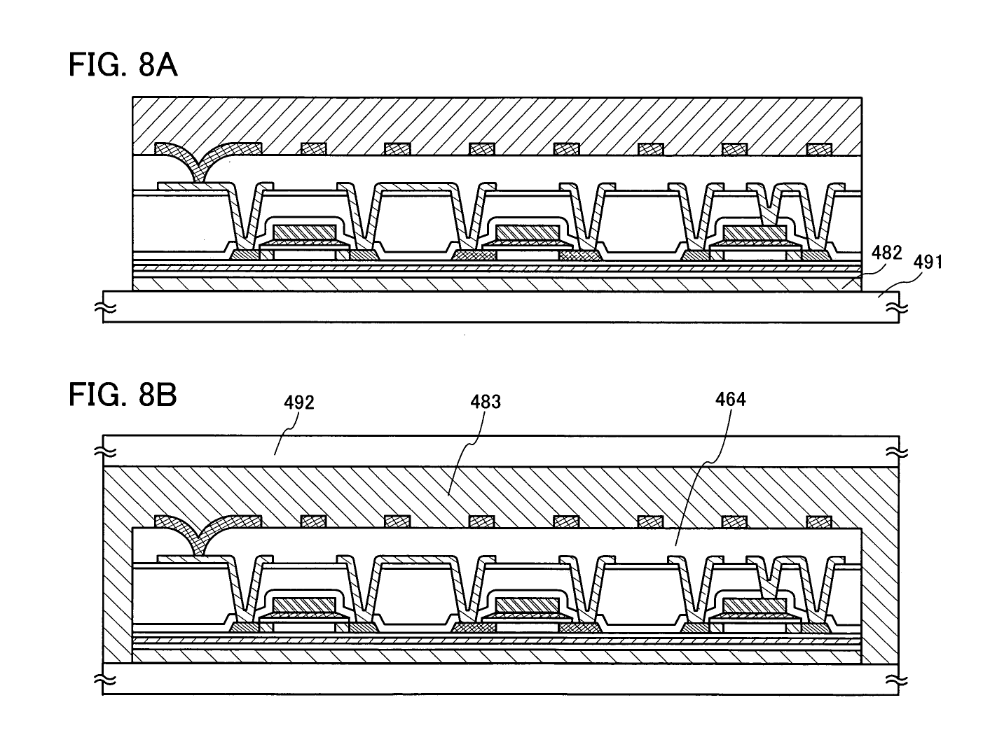

Subsequently, as shown in FIG. 8A, the TFTs 451, 452, and 453 separated from the first substrate 401 and the antenna 477 are attached to a second substrate 491 with the use of an adhesive 482. A material which can attach the second substrate 491 and the base film 403 to each other is used for the adhesive 482. As the adhesive 482, for example, various curable adhesives such as a reactive curable adhesive, a thermal curable adhesive, and a photo curable adhesive such as an ultraviolet curable adhesive, and an anaerobic adhesive can be used.

As the second substrate 491, a flexible organic material such as paper or plastic can be used. Alternatively, as the second substrate 491, a flexible inorganic material may be used. ARTON (manufactured by JSR) formed of poly norbornene having a polar group can be used as a plastic substrate. In addition, polyester typified by polyethylene terephthalate (PET); polyether sulfone (PES); polyethylene naphthalate (PEN); polycarbonate (PC); nylon; polyether etherketone (PEEK); polysulfone (PSF); polyether imide (PEI); polyarylate (PAR); polybutylene terephthalate (PBT); polyimide; an acrylonitrile butadiene styrene resin; poly vinyl chloride; polypropylene; poly vinyl acetate; an acrylic resin; and the like can be given. It is preferable that the second substrate 491 have high thermal conductivity of approximately 2 to 30 W/mK in order to disperse heat generated in an integrated circuit.

Next, as shown in FIG. 8B, after removing the protective layer 465, an adhesive 483 is applied on the third interlayer insulating film 464 so as to cover the antenna 477, whereby a cover material 492 is attached thereto. Similarly to the second substrate 491, a flexible organic material such as paper or plastic can be used as the cover material 492. For example, the thickness of the adhesive 483 may be 10 to 200 μm.

A material which can attach the cover material 492, the third interlayer insulating film 464, and the antenna 477 is used for the adhesive 483. As the adhesive 483, for example, various curable adhesives such as a reactive curable adhesive, a thermal curable adhesive, and a photo curable adhesive such as an ultraviolet curable adhesive, and an anaerobic adhesive can be used.

Through the above-described steps, the ID chip is completed. By the above-described formation method, an integrated circuit having a total thickness of greater than or equal to 0.3 μm and less than or equal to 3 μm, typically approximately 2 μm, which is considerably thin, can be formed between the second substrate 491 and the cover material 492. It is to be noted that the thickness of the integrated circuit includes the thicknesses of various insulating films and interlayer insulating films formed between the adhesive 482 and the adhesive 483 as well as the thickness of a semiconductor element itself. In addition, the integrated circuit included in the ID chip can be formed so as to occupy an area of less than or equal to 5 mm square (25 mm²), more desirably, approximately 0.3 mm square (0.09 mm²) to 4 mm square (16 mm²).

Further, when the integrated circuit is provided at a position between the second substrate 491 and the cover material 492 that is more centered, mechanical strength of the ID chip can be increased. Specifically, when the distance between the second substrate 491 and the cover material 492 is set to be d, it is desirable that the thicknesses of the adhesive 482 and the adhesive 483χ be controlled so that the distance between the second substrate 491 and the center in a direction of the thickness of the integrated circuit satisfies the following Formula 1.

$$\frac{1}{2} d - 30 μ m < χ < \frac{1}{2} d + 30 μ m \qquad [ Formula 1 ]$$

It is further preferable that the thicknesses of the adhesive 482 and the adhesive 483χ be controlled so that the following Formula 2 is satisfied.

$$\frac{1}{2} d - 10 μ m < χ < \frac{1}{2} d + 10 μ m \qquad [ Formula 2 ]$$

It is to be noted that the example in which the cover material 492 is used is shown in FIG. 8B; however, the present invention is not limited thereto. For example, the formation may be finished in the step shown in FIG. 8A.

The method in which the separation layer is provided between the first substrate 401 having high heat resistance and the integrated circuit, and the separation layer is removed by etching so that the substrate and the integrated circuit are separated from each other is shown in this embodiment; however, a manufacturing method of the ID chip of the present invention is not limited to this structure. For example, a metal oxide film may be provided between the substrate having high heat resistance and the integrated circuit, and the metal oxide film may be weakened by crystallization so that the integrated circuit is separated. Alternatively, a separation layer formed of an amorphous semiconductor film containing hydrogen may be provided between the substrate having high heat resistance and the integrated circuit, and the separation layer may be removed by laser light irradiation so that the substrate and the integrated circuit are separated from each other. Alternatively, the substrate having high heat resistance over which the integrated circuit is formed is mechanically removed or removed by etching with the use of a solution or a gas so that the integrated circuit may be separated from the substrate.

In the case of using an organic resin for the adhesive 482 which is in contact with the base film 403 in order to ensure flexibility of the ID chip, the use of a silicon nitride film or a silicon oxide film containing nitrogen as the base film 403 makes it possible to prevent an alkali metal such as Na or an alkaline earth metal from diffusing into the semiconductor film, from the organic resin.

In the case where a surface of an object is curved and therefore the second substrate 491 of the ID chip attached to the curved surface is curved so as to have a curved surface along movement of a generating line of a conical surface, a columnar surface, or the like, it is desirable to make a direction of the generating line and a moving direction of carriers of the TFTs 451, 452, and 453 be the same. The above-described structure makes it possible to suppress effects on electric characteristics of the TFTs 451, 452, and 453 even when the second substrate 491 is curved. In addition, when a ratio of an area occupied by the island-shaped semiconductor film in the integrated circuit is set to be 1 to 30%, effects on the electric characteristics of the TFTs 451, 452, and 453 can be further suppressed even when the second substrate 491 is curved.

In this embodiment, the example in which the antenna is formed over the same substrate as the integrated circuit is explained; however, the present invention is not limited to this structure. The antenna and the integrated circuit may be formed over different substrates and attached to each other afterward, so that they are electrically connected to each other.

Further, the frequency of an electric wave generally applied in the ID chip is 13.56 MHz or 2.45 GHz, and it is very important to form the ID chip so that the electric wave with the frequency can be detected, in order to enhance versatility.

The ID chip of this embodiment has a merit in that an electric wave is hardly blocked and a signal can be prevented from attenuating due to the blocked electric wave, in comparison with an ID chip formed using a semiconductor substrate. Therefore, the semiconductor substrate is not necessary to be used, and thus, costs of the ID chip can be drastically reduced. For example, the case of using a silicon substrate of 12 inches in diameter and the case of using a glass substrate with an area of 730 mm×920 mm are compared with each other. An area of the former silicon substrate is about 73000 mm² while an area of the latter glass substrate is about 672000 mm², and therefore, an area of the glass substrate is about 9.2 times as large as that of the silicon substrate. In the glass substrate with an area of about 672000 mm², about 672000 ID chips of 1 mm square are estimated to be produced if areas which are consumed by division of the substrate are not counted. The number of ID chips is about 9.2 times as large as that of the case of using the silicon substrate. Since the case of using the glass substrate with an area of 730 mm×920 mm requires smaller number of steps than the case of using the silicon substrate of 12 inches in diameter, investment costs in facilities for mass production of the ID chip can be reduced to one-third. Furthermore, according to the present invention, the glass substrate can be reused after separating the integrated circuit therefrom. Therefore, the costs can be drastically reduced in comparison with the case of using the silicon substrate even if costs for covering a cracked glass substrate or cleaning the surface of the glass substrate are taken into consideration. Moreover, if the glass substrate is not reused and is discarded, since the price of the glass substrate with an area of 730 mm×920 mm is about half of the price of the silicon substrate with 12 inches in diameter, the costs of manufacturing the ID chip can be drastically reduced.

Therefore, it is found that, in the case of using the glass substrate with an area of 730 mm×920 mm, the price of the ID chip can be suppressed to about one-thirtieth in comparison with the case of using the silicon substrate of 12 inches in diameter. The use of the ID chip as premises for a disposable one is considered, and thus, the ID chip of the present invention which can be formed with drastically reduced costs is very useful for the above-described purpose.

In this embodiment, the example in which the integrated circuit is separated to be attached to the flexible substrate is explained; however, the present invention is not limited to this structure. For example, in the case of using a substrate having allowable temperature limit which can resist heat treatment in manufacturing steps of the integrated circuit, such as glass substrate, the integrated circuit is not necessarily separated.

This embodiment can be freely combined with Embodiment Mode and other embodiments, if necessary.

### Embodiment 3

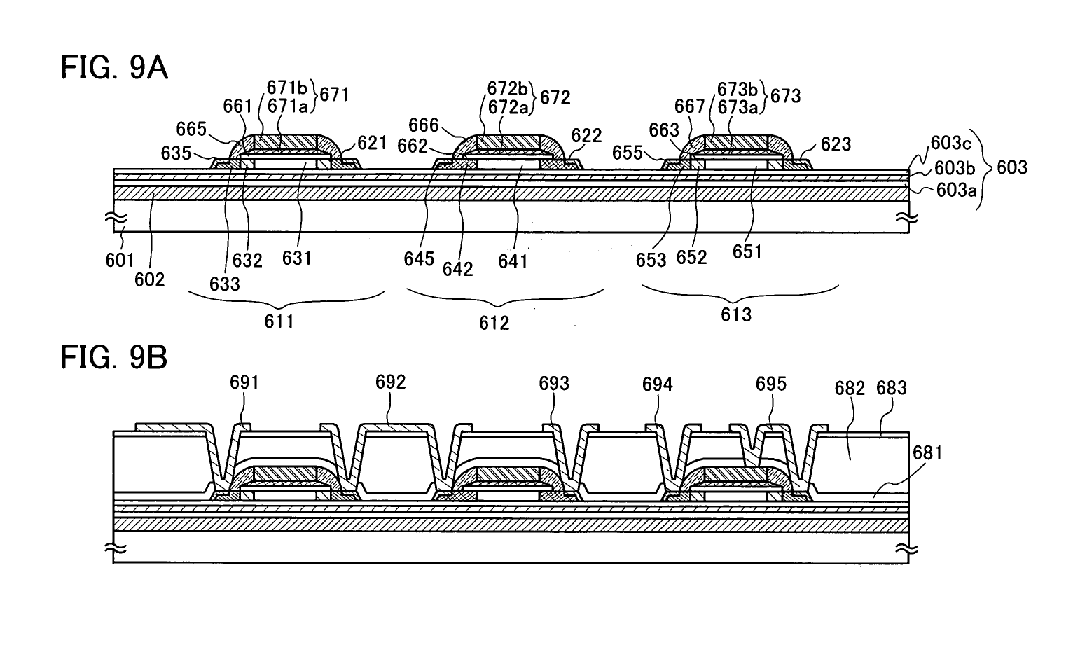

In this embodiment, an example of manufacturing an ID chip (also referred to as an IC chip or an IC tag) incorporated in a biological information detection sensor of the present invention which differs from that in Embodiment 2 will be explained with reference to FIGS. 9A and 9B, FIGS. 10A and 10B, FIGS. 11A and 11B, and FIGS. 12A and 12B.

In this embodiment, a TFT that is insulated and isolated is exemplified as a semiconductor element; however, a semiconductor element used for an integrated circuit is not limited thereto, and all circuit elements can be used. For example, a memory element, a diode, a photoelectric conversion element, a resistance element, a coil, a capacitor element, an inductor, and the like are typically given, besides a TFT.

First, a separation layer 602 is formed over a substrate (a first substrate) 601 having heat resistance by a sputtering method. For example, as the first substrate 601, a glass substrate such as barium borosilicate glass or aluminoborosilicate glass, a quartz substrate, a ceramic substrate, or the like can be used. Alternatively, a metal substrate including a stainless steel substrate or a semiconductor substrate over which an insulating film is formed may be used. A substrate formed of a synthetic resin having flexibility, such as plastic, generally tends to have lower allowable temperature limit than the above-described substrates; however, the substrate can be used as long as it can resist a processing temperature in manufacturing steps.

As the separation layer 602, a layer containing silicon such as amorphous silicon, polycrystalline silicon, single-crystal silicon, or microcrystalline silicon (including semi-amorphous silicon) as its main component can be used. The separation layer 602 can be formed by a sputtering method, a low-pressure CVD method, a plasma CVD method, or the like. In this embodiment, amorphous silicon formed with a thickness of approximately 50 nm by a low-pressure CVD method is used as the separation layer 602. Further, the separation layer 602 is not limited to silicon, and a material which can be selectively removed by etching may be used. It is desirable that the thickness of the separation layer 602 be 50 to 60 nm. If semi-amorphous silicon is used, the thickness thereof may be 30 to 50 nm.

It is to be noted that a semi-amorphous semiconductor typified by semi-amorphous silicon is a film containing a semiconductor having an intermediate structure between an amorphous semiconductor and a semiconductor having a crystalline structure (including single crystal and polycrystalline structures). This semi-amorphous semiconductor has a third state which is stable in terms of free energy, and which is crystalline having a short range order and lattice distortion. The semi-amorphous semiconductor can be formed with a grain diameter of 0.5 to 20 nm to be dispersed in a non single-crystal semiconductor. A Raman spectrum of the semi-amorphous silicon is shifted to a lower wavenumber than 520 cm⁻¹. The diffraction peaks of (111) and (220) which are thought to be derived from a Si crystalline lattice are observed by X-ray diffraction. Hydrogen or halogen of at least 1 atomic % or more is contained as a material for terminating dangling bonds. Such a semiconductor is referred to as a semi-amorphous semiconductor (SAS) for the sake of convenience. A favorable semi-amorphous semiconductor with increased stability can be obtained by further promotion of the lattice distortion by means of adding a rare gas element such as helium, argon, krypton, or neon.

Semi-amorphous silicon can be obtained by glow discharge decomposition of a gas containing silicon. As the typical gas containing silicon, SiH₄ is given, and besides, Si₂H₆, SiH₂Cl₂, SiHCl₃, SiCl₄, SiF₄, or the like can be used. Semi-amorphous silicon can be easily formed with the use of the gas containing silicon diluted with hydrogen or a gas in which one or more of rare gas elements selected from helium, argon, krypton, and neon is/are added to hydrogen. It is preferable to dilute the gas containing silicon at a dilution rate in the range of 2 to 1000 times.

Subsequently, a base film 603 is formed over the separation layer 602. The base film 603 is provided to prevent an alkali metal such as Na or an alkaline earth metal contained in the first substrate 601 from diffusing into a semiconductor film and having an adverse effect on an electric characteristic of a semiconductor element such as a TFT. In addition, the base film 603 also has a role of protecting the semiconductor element in a later step of separating the semiconductor element. The base film 603 may be a single layer or a stacked layer of a plurality of insulating films. Consequently, the base film 603 is formed using an insulating film such as silicon oxide, silicon nitride, silicon oxide containing nitrogen, silicon nitride containing oxygen, or the like, which can suppress diffusion of an alkali metal or an alkaline earth metal into a semiconductor film.

In this embodiment, a silicon oxide film containing nitrogen with a thickness of 100 nm as a lower base film 603a, a silicon nitride film containing oxygen with a thickness of 50 nm as a middle base film 603b, and a silicon oxide film containing nitrogen with a thickness of 100 nm as an upper base film 603c are stacked in this order to form the base film 603. However, a material and a thickness of each film, and the number of stacked layers are not limited thereto. For example, instead of the silicon oxide film containing nitrogen, which is the lower base film 603a, a siloxane-based resin may be formed with a thickness of 0.5 to 3 μm by a spin coating method, a slit coating method, a droplet discharging method, or the like. Instead of the silicon nitride film containing oxygen, which is the middle base film 603b, a silicon nitride film (Si₃N₄ or the like) may be used. Instead of the silicon oxide film containing nitrogen, which is the upper base film 603c, a silicon oxide film may be used. It is desirable that the thickness of each film be 0.05 to 3 μm, and the thickness can be freely selected from this range.

Alternatively, the lower base film 603a of the base film 603, which is closest to the separation layer 602, may be formed of a silicon oxide film containing nitrogen or a silicon oxide film, the middle base film 603b may be formed of a siloxane-based resin, and the upper base film 603c may be formed of a silicon oxide film.

Here, the silicon oxide film can be formed using a mixed gas of SiH₄ and O₂, TEOS (tetraethoxysilane) and O₂, or the like, by a method such as thermal CVD, plasma CVD, normal pressure CVD, or bias ECRCVD. Also, the silicon nitride film can be formed typically using a mixed gas of SiH₄ and NH₃, by plasma CVD. Furthermore, the silicon oxide film containing nitrogen (composition ratio O>N) and the silicon nitride film containing oxygen (composition ratio N>O) can be formed typically using a mixed gas of SiH₄ and N₂O, by plasma CVD.

Subsequently, a semiconductor film is formed over the base film 603. It is preferable that, after forming the base film 603, the semiconductor film be formed without exposure to the air. The thickness of the semiconductor film is 20 to 200 nm (desirably, 40 to 170 nm, preferably, 50 to 150 nm). Further, the semiconductor film may be an amorphous semiconductor, a semi-amorphous semiconductor, or a polycrystalline semiconductor. Not only silicon but also silicon germanium can be used as the semiconductor. In the case of using silicon germanium, a concentration of the germanium is preferably about 0.01 to 4.5 atomic %.

The amorphous semiconductor can be obtained by glow discharge decomposition of a gas containing silicon. As the typical gas containing silicon, SiH₄, and Si₂H₆ are given. Those gases containing silicon may be used by dilution with hydrogen, or hydrogen and helium.

As described above, the semi-amorphous semiconductor can be obtained by glow discharge decomposition of the gas containing silicon. However, a carbide gas such as CH₄ or C₂H₆, a germanium gas such as GeH₄ or GeF₄, or F₂ may be mixed into the gas containing silicon to adjust the width of the energy band to 1.5 to 2.4 eV or 0.9 to 1.1 eV.

For example, in the case of using a gas in which H₂ is added to SiH₄ or the gas in which F₂ is added to SiH₄, the subthreshold coefficient (S value) of the TFT can be less than or equal to 0.35 V/dec, typically, 0.25 to 0.09 V/dec, and the mobility can be 10 cm²/Vs when the TFT is manufactured using the formed semi-amorphous semiconductor. When 19-stage ring oscillator is formed of a TFT using the above-described semi-amorphous semiconductor, for example, a property in which a repetition rate is greater than or equal to 1 MHz, preferably, greater than or equal to 100 MHz, with power supply voltage of 3 to 5V, can be obtained. In addition, in the power supply voltage of 3 to 5V, delay time per one stage of an inverter can be 26 ns, preferably, less than or equal to 0.26 ns.

Next, crystallization is performed by irradiation of the semiconductor film with a linear beam from a laser irradiation apparatus.

In the case of performing laser crystallization, before performing the laser crystallization, heat treatment at 500° C. for 1 hour may be performed to the semiconductor film in order to increase resistance of the semiconductor film to the laser.

For the laser crystallization, a continuous wave laser (CW laser) can be used, or alternatively, a pulse oscillation laser at a repetition rate of greater than or equal to 10 MHz, preferably, greater than or equal to 80 MHz can be used as a pseudo CW laser.

Specifically, the following can be given as the continuous wave laser: an Ar laser, a Kr laser, a CO₂ laser, a YAG laser, a YVO₄ laser, a forsterite (Mg₂SiO₄) laser, a YLF laser, a YAlO₃ laser, a GdVO₄ laser, a Y₂O₃ laser, an alexandrite laser, a Ti:sapphire laser, a helium cadmium laser, and a laser of which a medium is a polycrystalline (ceramic) YAG, Y₂O₃, YVO₄, YAlO₃, or GdVO₄ doped with one or more of Nd, Yb, Cr, Ti, Ho, Er, Tm, and Ta as a dopant.

As the pseudo CW laser, the following can be used as long as pulse oscillation with a repetition rate of greater than or equal to 10 MHz, preferably, greater than or equal to 80 MHz is possible: the pulse oscillation laser such as an Ar laser, a Kr laser, an excimer laser, a CO₂ laser, a YAG laser, a Y₂O₃ laser, a YVO₄ laser, a forsterite (Mg₂SiO₄) laser, a YLF laser, a YAlO₃ laser, a GdVO₄ laser, an alexandrite laser, a Ti:sapphire laser, a copper vapor laser, a gold vapor laser, or a laser of which a medium is a polycrystalline (ceramic) YAG, Y₂O₃, YVO₄, YAlO₃, or GdVO₄ doped with one or more of Nd, Yb, Cr, Ti, Ho, Er, Tm, and Ta as a dopant.

Such a pulse oscillation laser eventually shows an effect equivalent to that of a continuous wave laser when the repetition rate is increased.

For example, in the case of using a solid-state laser capable of continuous oscillation, a crystal with a large grain diameter can be obtained by irradiation with laser light of the second to fourth harmonics. Typically, it is desirable to use the second harmonic (532 nm) or the third harmonic (355 nm) of the YAG laser (fundamental wave of 1064 nm). For example, laser light emitted from a continuous wave YAG laser is converted into a high harmonic by a nonlinear optical element, and emitted to the semiconductor film. An energy density may be approximately 0.01 to 100 MW/cm² (preferably 0.1 to 10 MW/cm²). Then, irradiation is carried out at a scanning speed of approximately 10 to 2000 cm/s.

It is to be noted that a laser of which a medium is a single crystal YAG; YVO₄, forsterite (Mg₂SiO₄), YAlO₃, or GdVO₄ doped with one or more of Nd, Yb, Cr, Ti, Ho, Er, Tm, and Ta as a dopant, or a polycrystalline (ceramic) YAG, Y₂O₃, YVO₄, YAlO₃, or GdVO₄ doped with one or more of Nd, Yb, Cr, Ti, Ho, Er, Tm, and Ta as a dopant; an Ar laser; a Kr laser; or a Ti:sapphire laser is capable of continuous oscillation, and is also capable of pulse oscillation by a Q switch operation, mode locking, or the like. When a laser beam is oscillated at a repetition rate of greater than or equal to 10 MHz, the semiconductor film is irradiated with a subsequent pulse while the semiconductor film is melted by a preceding laser and then solidified. Consequently, since a solid-liquid interface in the semiconductor film can be moved continuously unlike in the case of using a pulse laser with a low repetition rate, crystal grains that continuously grow towards a scanning direction can be obtained.

When a ceramic (a poly crystal) is used for a medium, the medium can be formed into a free shape in a short amount of time and at low costs. In the case of using a single crystal, a column-shaped medium with several mm in diameter and several ten mm in length is usually used; however, a larger medium can be formed in the case of using ceramic.

Since a concentration of a dopant such as Nd or Yb in the medium which directly contributes to light emission cannot be changed significantly in either a single crystal or a poly crystal, improvement in laser output by increase of the concentration is limited to a certain extent. However, in the case of ceramic, there is a possibility that output can be drastically improved since the size of the medium can be significantly increased in comparison with a single crystal.

Furthermore, in the case of ceramic, a medium having a parallelepiped shape or a rectangular parallelepiped shape can be easily formed. When a medium having such a shape is used and oscillation light goes in zigzag in the medium, an oscillation light path can be longer. Accordingly, amplification is increased and oscillation with high output is possible. Since a laser beam emitted from the medium having such a shape has a cross section of a quadrangular shape when being emitted, a linear beam can be easily shaped in comparison with the case of a circular beam. The laser beam emitted in such a manner is shaped with the use of an optical system; accordingly, a linear beam having a short side of less than or equal to 1 mm and a long side of several mm to several m can be easily obtained. In addition, by uniform irradiation of the medium with excited light, the linear beam has a uniform energy distribution in a long side direction.

By irradiation of the semiconductor film with this linear beam, an entire surface of the semiconductor film can be annealed more uniformly. In the case where uniform annealing is necessary from one edge to the other edge of the linear beam, slits may be provided for both edges so as to shield a portion where energy is attenuated from light.

By irradiation of the semiconductor film with the above-described laser light, a crystalline semiconductor film with further increased crystallinity is formed.

Subsequently, island-shaped semiconductor films 621, 622, and 623 are formed using the obtained crystalline semiconductor film. Each of the island-shaped semiconductor films becomes an active layer of a TFT to be formed in later steps.

Next, an impurity for controlling a threshold value is introduced into the island-shaped semiconductor film. In this embodiment, boron (B) is introduced into the island-shaped semiconductor film by doping of diborane (B₂H₆).

Then, an insulating film is formed so as to cover the island-shaped semiconductor films 621, 622, and 623. For example, silicon oxide, silicon nitride, silicon oxide containing nitrogen, or the like can be used for the insulating film. As a formation method, a plasma CVD method, a sputtering method, or the like can be used.

Next, gate insulating films 661, 662, and 663 are formed using an insulating film over the island-shaped semiconductor films 621, 622, and 623, respectively.

A first conductive film and a second conductive film are formed so as to cover the island-shaped semiconductor films 621, 622, and 623 and the gate insulating films 661, 662, and 663.

The first conductive film and the second conductive film are formed of an element selected from tantalum (Ta), tungsten (W), titanium (Ti), molybdenum (Mo), and aluminum (Al), or an alloy material or compound material containing the above-described element as its main component.

In this embodiment, for example, a tantalum nitride (TaN) film is formed with a thickness of 10 to 50 nm, for example, 30 nm as the first conductive film. Then, over the first conductive film, for example, a tungsten (W) film is formed with a thickness of 200 to 400 nm, for example, 370 nm as the second conductive film; accordingly, the stacked film of the first conductive film and the second conductive film is formed.

Subsequently, the second conductive film is etched by anisotropic etching to form upper gate electrodes 671b, 672b, and 673b. Then, the first conductive film is etched by isotropic etching to form lower gate electrodes 671a, 672a, and 673a. Accordingly, gate electrodes 671, 672, and 673 are formed.

The gate electrodes 671, 672, and 673 may be used as part of a gate wiring. Alternatively, another gate wiring may be formed to connect the gate electrodes 671, 672, and 673 thereto.

Subsequently, a resist is formed so as to cover the island-shaped semiconductor film 622 which becomes an active layer of a p-channel TFT 612, the gate insulating film 662, and the gate electrode 672.

Then, an impurity imparting n-type conductivity is added to the island-shaped semiconductor film 621 with the use of the gate electrode 671 and the gate insulating film 661 as masks, so that a channel formation region 631, a low concentration impurity region 632, and a source or drain region 633 are formed. In addition, at the same time, an impurity imparting n-type conductivity is added to the island-shaped semiconductor film 623 with the use of the gate electrode 673 and the gate insulating film 663 as masks, so that a channel formation region 651, a low concentration impurity region 652, and a source or drain region 653 are formed.

First, with the use of phosphine (PH₃), phosphorus (P), which is used as the impurity imparting n-type conductivity, is introduced into the island-shaped semiconductor films 621 and 623 with acceleration voltage of 40 to 100 keV, for example, 60 keV, and a dose amount of 1×10¹³ to 1×10¹⁵ cm⁻², for example, 2.6×10¹³ cm⁻². When introducing the impurity, the channel formation region 631 of an n-channel TFT 611 and the channel formation region 651 of an n-channel TFT 613 are formed.

Subsequently, with the use of phosphine (PH₃), phosphorus (P) is introduced into the island-shaped semiconductor films 621 and 623 with acceleration voltage of 10 to 60 keV, for example, 20 keV, and a dose amount of 5.0×10¹⁴ to 2.5×10¹⁶ cm⁻², for example, 3.0×10¹⁵ cm². Accordingly, the low concentration impurity region 632 and the source or drain region 633 of the n-channel TFT 611 are formed. In addition, the low concentration impurity region 652 and the source or drain region 653 of the n-channel TFT 613 are formed.

In this embodiment, phosphorus (P) is contained in each of the source or drain region 633 of the n-channel TFT 611 and the source or drain region 653 of the n-channel TFT 613 at a concentration of 1×10¹⁹ to 5×10²¹ cm⁻³. In addition, phosphorus (P) is contained in each of the low concentration impurity region 632 of the n-channel TFT 611 and the low concentration impurity region 652 of the n-channel TFT 613 at a concentration of 1×10¹⁸ to 5×10¹⁹ cm⁻³.

Subsequently, the resist over the island-shaped semiconductor film 622, the gate insulating film 662, and the gate electrode 672 is removed, and a resist is formed so as to cover the island-shaped semiconductor film 621, the gate insulating film 661, and the gate electrode 671 and a resist is formed so as to cover the island-shaped semiconductor film 623, the gate insulating film 663, and the gate electrode 673.

In addition, in order to manufacture a p-channel TFT 612, boron (B) is introduced into the island-shaped semiconductor film 622 with the use of diborane (B₂H₆) with applied voltage of 60 to 100 keV, for example, 80 keV, and a dose amount of 1×10¹³ to 5×10¹⁵ cm⁻², for example, 3×10¹⁵ cm⁻². Accordingly, a source region or drain region 642 of the p-channel TFT 612 is formed, and furthermore, a channel formation region 641 is formed when introducing the impurity.

Further, in the p-channel TFT 612, since the applied voltage is high when introducing boron, boron enough for forming the source or drain region 642 is added to the island-shaped semiconductor film 622, even if the boron passes through the lower gate electrode 672a and the gate insulating film 662.

Boron (B) is contained in the source or drain region 642 of the p-channel TFT 612 at a concentration of 1×10¹⁹ to 5×10²¹ cm⁻³.

Subsequently, after removing the resist, an insulating film for forming a sidewall is formed so as to cover the island-shaped semiconductor films 621, 622, and 623, the gate insulating films 661, 662, and 663, and the gate electrodes 671, 672, and 673.

This insulating film can be formed using a silicon oxide film or a silicon oxide film containing nitrogen by a plasma CVD method or a low-pressure CVD (LPCVD) method. In this embodiment, a silicon oxide film is formed with a thickness of 50 to 200 nm, preferably, 100 nm by a plasma CVD method.

Then, the insulating film is etched to form a sidewall 665 on side surfaces of the gate insulating film 661 and the gate electrode 671, a sidewall 666 on side surfaces of the gate insulating film 662 and the gate electrode 672, and a sidewall 667 on side surfaces of the gate insulating film 663 and the gate electrode 673. The sidewalls 665, 666, and 667 are formed so as to be tapered or rectangular. In this embodiment, the tapered sidewalls 665, 666, and 667 are formed.

Subsequently, a metal film is formed so as to cover the island-shaped semiconductor films 621, 622, and 623, the gate insulating films 661, 662, and 663, the gate electrodes 671, 672, and 673, and the sidewalls 665, 666, and 667.

Titanium (Ti), nickel (Ni), cobalt (Co), tungsten (W), platinum (Pt), or the like can be used for the metal film. In this embodiment, a nickel film is formed with a thickness of 10 nm as the metal film.

Then, the island-shaped semiconductor films 621, 622, and 623 to which the metal film is formed is heated by a thermal annealing method using an annealing furnace, a laser annealing method, or a rapid thermal annealing method (RTA method). Accordingly, a silicide region 635 is formed in the island-shaped semiconductor film 621, a silicide region 645 is formed in the island-shaped semiconductor film 622, and a silicide region 655 is formed in the island-shaped semiconductor film 623. In this embodiment, the silicide regions 635, 645, and 655 are formed by heating at a temperature of greater than or equal to 350° C. by a rapid thermal annealing method.

After forming the silicide regions 635, 645, and 655, an unreacted metal film is removed by etching by a chemical such as sulfuric acid or nitric acid.

Accordingly, the n-channel TFT 611, the p-channel TFT 612, and the n-channel TFT 613 are formed (see FIG. 9A). It is to be noted that each of the TFTs 611, 612, and 613 has a top gate structure in this embodiment; however, a bottom gate structure (an inversely staggered structure) may be employed.

The n-channel TFT 611 has the island-shaped semiconductor film 621, the gate insulating film 661, and the gate electrode 671 including the lower gate electrode 671a and the upper gate electrode 671b, over the upper base film 603c. The channel formation region 631, the low concentration impurity region 632, the source or drain region 633, and the silicide region 635 are formed in the island-shaped semiconductor film 621. The silicide region 635 is formed in part of the source or drain region 633. In addition, the sidewall 665 is formed on the side surfaces of the gate insulating film 661 and the gate electrode 671.

The p-channel TFT 612 has the island-shaped semiconductor film 622, the gate insulating film 662, and the gate electrode 672 including the lower gate electrode 672a and the upper gate electrode 672b, over the upper base film 603c. The channel formation region 641, the source or drain region 642, and the silicide region 645 are formed in the island-shaped semiconductor film 622. The silicide region 645 is formed in part of the source or drain region 642. In addition, the sidewall 666 is formed on the side surfaces of the gate insulating film 662 and the gate electrode 672.

The n-channel TFT 613 has the island-shaped semiconductor film 623, the gate insulating film 663, and the gate electrode 673 including the lower gate electrode 673a and the upper gate electrode 673b, over the upper base film 603c. The channel formation region 651, the low concentration impurity region 652, the source or drain region 653, and the silicide region 655 are formed in the island-shaped semiconductor film 623. The silicide region 655 is formed in part of the source or drain region 653. In addition, the sidewall 667 is formed on the side surfaces of the gate insulating film 663 and the gate electrode 673.

Thereafter, a passivation film 681 for protecting the TFTs 611, 612, and 613 may be further formed. It is preferable that the passivation film 681 be formed using silicon nitride, silicon oxide containing nitrogen, aluminum nitride, aluminum oxide, silicon oxide, or the like which can prevent an alkali metal or an alkaline earth metal from entering the TFTs 611, 612, and 613. Specifically, for example, a silicon oxide film containing nitrogen with a thickness of approximately 600 nm can be used as the passivation film. In this case, a hydrogenation treatment step may be performed after forming the silicon oxide film containing nitrogen. By the above-described structure, the TFTs 611, 612, and 613 are covered with the base film 603 and the passivation film 681; therefore, an alkali metal such as Na or an alkaline earth metal can be prevented from diffusing into the semiconductor film used for a semiconductor element and having an adverse effect on an electric characteristic of the semiconductor element.

Subsequently, a first interlayer insulating film 682 is formed so as to cover the TFTs 611, 612, and 613 and the passivation film 681. A heat resistant organic resin such as polyimide, acrylic, or polyamide can be used for the first interlayer insulating film 682. Besides the above-described organic resin, a low-dielectric material (a low-k material), a resin including a Si—O—Si bond formed using a siloxane-based material as a starting material (hereinafter, referred to as a siloxane-based resin), or the like can be used.

Siloxane has a skeleton structure formed by a bond of silicon (Si) and oxygen (O) and has an organic group containing at least hydrogen (for example, an alkyl group or an aryl group) as a substituent. Alternatively, as the substituent, a fluoro group may be used. Further alternatively, as the substituent, an organic group containing at least hydrogen and a fluoro group may be used.

The first interlayer insulating film 682 can be formed by spin coating, dipping, spray coating, droplet discharging (ink jetting, screen printing, offset printing, or the like), a doctor knife, a roll coater, a curtain coater, a knife coater, or the like, depending on the material thereof. Alternatively, the first interlayer insulating film 682 can be formed using an inorganic material such as silicon oxide, silicon nitride, silicon oxynitride, PSG (phosphosilicate glass), BPSG (boron phosphosilicate glass), an alumina film, or the like. Further, these insulating films may be stacked to form the first interlayer insulating film 682.

Furthermore, in this embodiment, a second interlayer insulating film 683 is formed over the first interlayer insulating film 682. As the second interlayer insulating film 683, a film containing carbon such as DLC (diamond like carbon) or carbon nitride (CN), a silicon oxide film, a silicon nitride film, a silicon oxide film containing nitrogen, or the like can be used. As a formation method, a plasma CVD method, an atmospheric pressure plasma, or the like can be used. Alternatively, a photosensitive or nonphotosensitive organic material such as polyimide, acrylic, polyamide, resist, or benzocyclobutene; a siloxane-based resin; or the like may be used.

Filler may be mixed into the first interlayer insulating film 682 or the second interlayer insulating film 683 in order to prevent the first interlayer insulating film 682 or the second interlayer insulating film 683 from being peeled off or cracked due to stress generated by a difference in coefficient of thermal expansion between the first interlayer insulating film 682 or the second interlayer insulating film 683 and a conductive material for forming a wiring that is formed later, or the like.

Subsequently, contact holes are formed in the first interlayer insulating film 682 and the second interlayer insulating film 683. Then, electrodes or wirings 691, 692, 693, 694, and 695 which are connected to the TFTs 611, 612, and 613 through the contact holes are formed. In this embodiment, the electrode and the wiring are formed together; however, the electrode and the wiring may be formed separately and electrically connected to each other. Although a mixed gas of CHF₃ and He is used for etching when forming the contact holes, a gas is not limited thereto. In this embodiment, each of the electrodes or wirings 691, 692, 693, 694, and 695 is formed of a five-layered structure in which a titanium (Ti) film, a titanium nitride (TiN) film, an aluminum containing silicon (Al—Si) film, a titanium (Ti) film, and a titanium nitride (TiN) film are stacked by a sputtering method.

When silicon (Si) is mixed into an aluminum (Al) film, a hillock can be prevented from generating in resist baking when the wiring is formed. Instead of Si, copper (Cu) of approximately 0.5% may be mixed. In addition, when an Al—Si layer is interposed between titanium (Ti) and titanium nitride (TiN), suppressing hillock development is further improved. Further, it is desirable to use the above-described hard mask formed of silicon oxide containing nitrogen, or the like in etching. It is to be noted that a material or a formation method of the wiring are not limited to these, and the above-described material used for the gate electrode may also be used.

Alternatively, the electrodes or wirings 691, 692, 693, 694, and 695 may be formed of an aluminum alloy film containing carbon and at least one kind of element selected from nickel, cobalt, and iron. Such an aluminum alloy film can prevent interdiffusion of silicon and aluminum even if the electrodes or wirings 691, 692, 693, 694, and 695 are in contact with silicon. Also, with such an aluminum alloy film, oxidation-reduction reaction does not occur even if the aluminum alloy film is in contact with a transparent conductive film, for example, an ITO (indium tin oxide) film, and thus, they can be in direct contact with each other. Furthermore, such an aluminum alloy film has low resistivity and is excellent in heat resistance, and therefore, it is useful as a wiring material.

It is to be noted that the electrode or wiring 691 and the electrode or wiring 692 are connected to the silicide region in the source or drain region 633 of the n-channel TFT 611. The electrode or wiring 692 and the electrode or wiring 693 are electrically connected to the silicide region in the source or drain region 642 of the p-channel TFT 612. The electrode or wiring 694 and the electrode or wiring 695 are electrically connected to the silicide region in the source or drain region 653 of the n-channel TFT 613. Furthermore, the electrode or wiring 695 is also connected to the gate electrode 673 of the n-channel TFT 613. The n-channel TFT 613 can be used as a memory element of a random ROM (see FIG. 9B).

Subsequently, a third interlayer insulating film 701 is formed over the second interlayer insulating film 683 so as to cover the electrodes or wirings 691, 692, 693, 694, and 695. The third interlayer insulating film 701 is formed so as to have an opening at a position where part of the electrode or wiring 691 is exposed. Further, the third interlayer insulating film 701 can be formed using a material similar to that of the first interlayer insulating film 682.

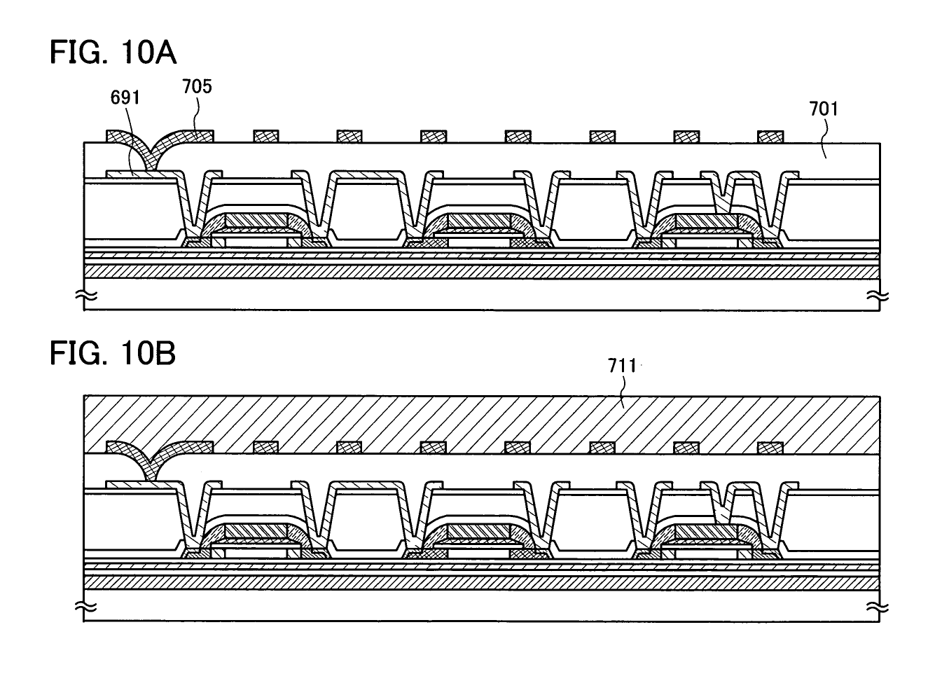

Next, an antenna 705 is formed over the third interlayer insulating film 701 (see FIG. 10A). The antenna 705 can be formed using a conductive material including one or more of metals such as Ag, Au, Cu, Pd, Cr, Mo, Ti, Ta, W, Al, Fe, Co, Zn, Sn, and Ni, and metal compounds thereof. The antenna 705 is connected to the electrode or wiring 691. It is to be noted that, in FIG. 10A, the antenna 705 is directly connected to the electrode or wiring 691; however, a structure of the ID chip of the present invention is not limited thereto. For example, the antenna 705 and the electrode or wiring 691 may be electrically connected to each other with the use of a wiring which is separately formed.

The antenna 705 can be formed by a printing method, a photolithography method, an evaporation method, a droplet discharging method, or the like. In this embodiment, the antenna 705 is formed of a single conductive film; however, the antenna 705 in which a plurality of conductive films is stacked can also be formed. For example, the antenna 705 may be formed in such a manner that a wiring formed of Ni or the like is coated with Cu by electroless plating.

It is to be noted that a droplet discharging method means a method in which droplets containing predetermined composition are discharged from a pore to form a predetermined pattern, and an ink jetting method is included in the category. Also, a screen printing method, an offset printing method, or the like is included in a printing method. The use of a printing method and a droplet discharging method makes it possible to form the antenna 705 without using a mask for exposure. In addition, when a droplet discharging method and a printing method are used, waste of material due to removal by etching does not occur, unlike a photolithography method. Moreover, an expensive mask for exposure is not necessary to be used; therefore, costs spent for manufacturing the ID chip can be reduced.

In the case of using a droplet discharging method or various printing methods, for example, a conductive particle in which Cu is coated with Ag, or the like can also be used. Further, in the case of forming the antenna 705 by a droplet discharging method, it is desirable that treatment for increasing adhesiveness of the antenna 705 be performed to a surface of the third interlayer insulating film 701.

As the method which can increase the adhesiveness, for example, the following can be specifically given: a method in which a metal or metal compound which can increase adhesiveness of a conductive film or an insulating film by catalysis is attached to the surface of the third interlayer insulating film 701, a method in which an organic insulating film, a metal, and a metal compound having high adhesiveness with a conductive film or insulating film to be formed are attached to the surface of the third interlayer insulating film 701, a method in which plasma treatment is performed to the surface of the third interlayer insulating film 701 under high atmospheric pressure or reduced pressure to perform surface modification, or the like. As above-described metal having high adhesiveness with the conductive film or insulating film, Sc, Ti, V, Cr, Mn, Fe, Co, Ni, Cu, Zn, and the like which are 3d transition elements can be given, besides titanium or titanium oxide. As the metal compound, oxide, nitride, oxynitride of the above-described metal, or the like can be given. As the above-described organic insulating film, for example, polyimide, a siloxane-based resin, or the like can be given.

In the case where the metal or metal compound to be attached to the third interlayer insulating film 701 has conductivity, sheet resistance thereof is controlled so that normal performance of the antenna is not disturbed. Specifically, it is preferable that an average thickness of the metal or metal compound having conductivity be controlled so as to be, for example, 1 to 10 nm, or part or all of the metal or metal compound be insulated by oxidation. Alternatively, the attached metal or metal compound in a region except for a region where the adhesiveness is desired to be increased may be selectively removed by etching. Alternatively, the metal or metal compound may be selectively attached only to a specific region by a droplet discharging method, a printing method, a sol-gel method, or the like without attaching the metal or metal compound on an entire surface of a substrate in advance. It is to be noted that the metal or metal compound is not necessarily in a completely continuous film form and may be dispersed to some extent on the surface of the third interlayer insulating film 701.

Then, as shown in FIG. 10B, after forming the antenna 705, a protective layer 711 is formed over the third interlayer insulating film 701 so as to cover the antenna 705. The protective layer 711 is formed using a material which can protect the antenna 705 when the separation layer 602 is removed by etching later. For example, the protective layer 711 can be formed by application of an epoxy-based resin, an acrylate-based resin, or a resin containing silicon that is soluble in water or alcohols over the entire surface.

In this embodiment, the protective layer 711 is formed in the following manner: a water-soluble resin (manufactured by Toagosei Co., Ltd.: VL-WSHL10) is applied so as to have a thickness of 30 μm by a spin coating method, is exposed to light for 2 minutes for temporary curing, and then, its back surface is exposed to UV light for 2.5 minutes and a front surface is exposed to the UV light for 10 minutes, 12.5 minutes in total, so that the resin is fully cured. Further, in the case of stacking a plurality of organic resins, the stacked organic resins might be partly melted depending on a solvent to be used, during application or baking, or the adhesiveness might become too high. Therefore, in the case of forming both the third interlayer insulating film 701 and the protective layer 711 using an organic resin that is soluble in the same solvent, it is preferable to form an inorganic insulating film (a silicon nitride film, a silicon nitride film containing oxygen, a silicon oxide film containing nitrogen, an aluminum nitride film, an aluminum nitride film containing oxygen, or an aluminum oxide film containing nitrogen) so as to cover the third interlayer insulating film 701 for smoothly removing the protective layer 711 in a later step.

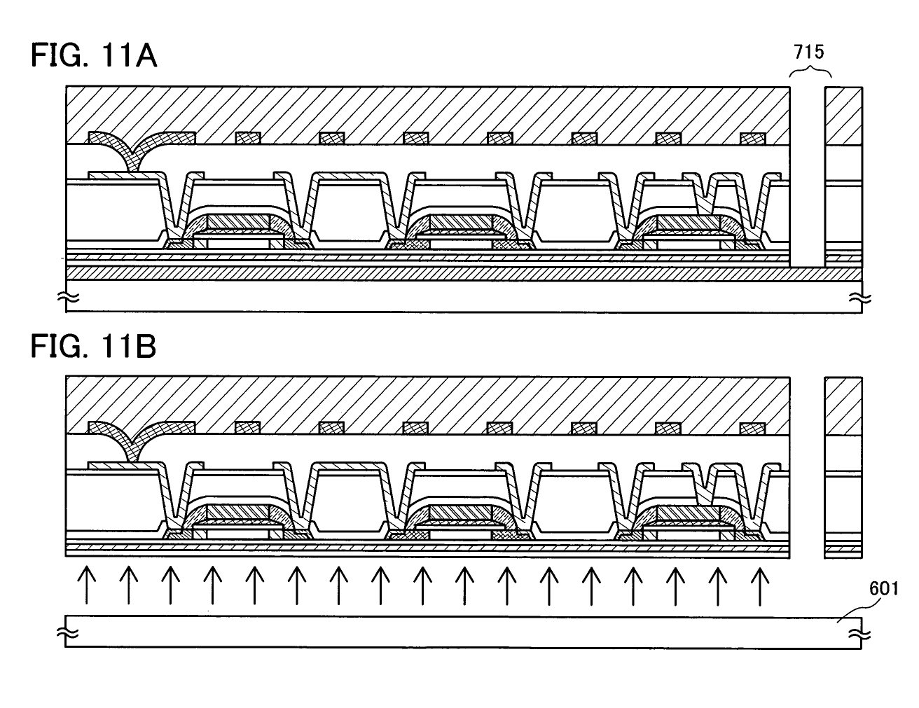

Subsequently, as shown in FIG. 11A, an opening 715 is formed in order to separate the ID chips. The opening 715 is formed to the extent that the separation layer 602 is exposed. The opening 715 can be formed by dicing, scribing, or the like. Further, in the case where the ID chips formed over the first substrate 601 are not necessary to be separated, the opening 715 is not necessarily formed.

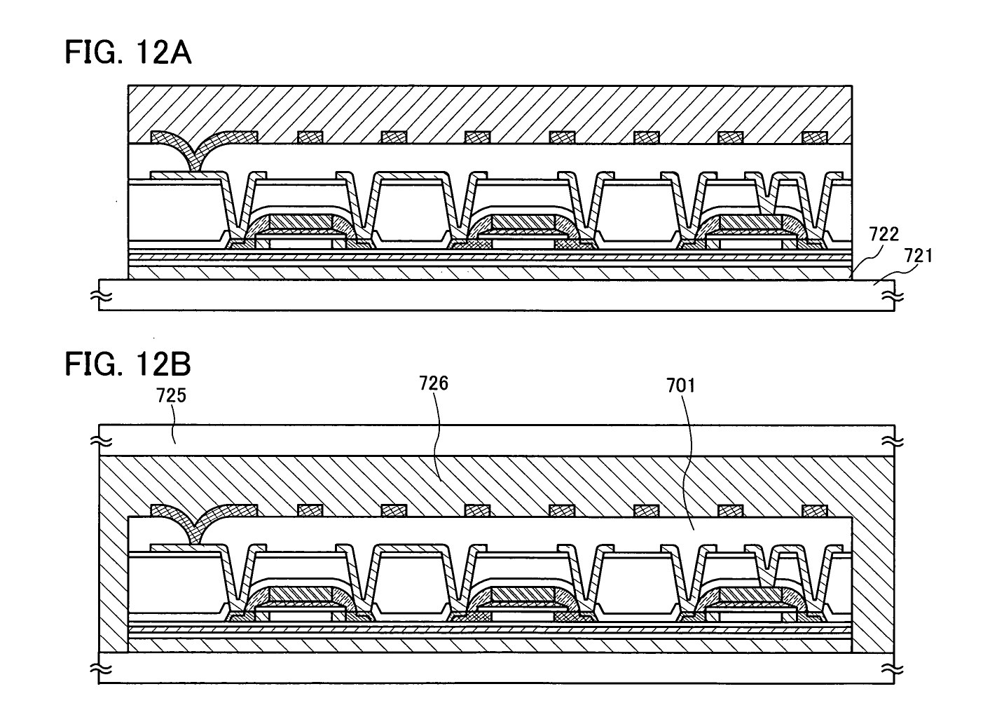

Next, as shown in FIG. 12B, the separation layer 602 is removed by etching. In this embodiment, halogen fluoride is used as an etching gas and the gas is introduced from the opening 715. In this embodiment, for example, the etching is performed using ClF₃ (chlorine trifluoride) at a temperature of 350° C. with a flow rate of 300 sccm and air pressure of 798 Pascal (798 Pa) for 3 hours. Alternatively, a gas in which nitrogen is mixed into a ClF₃ gas may be used. When halogen fluoride such as ClF₃ is used, the separation layer 602 is selectively etched and the first substrate 601 can be separated from the TFTs 611, 612, and 613. Further, the halogen fluoride may be either a gas or a liquid.

Subsequently, as shown in FIG. 12A, the TFTs 611, 612, and 613 separated from the first substrate 601 and the antenna 705 are attached to a second substrate 721 with the use of an adhesive 722. A material which can attach the second substrate 721 and the base film 603 to each other is used for the adhesive 722. As the adhesive 722, for example, various curable adhesives such as a reactive curable adhesive, a thermal curable adhesive, and a photo curable adhesive such as an ultraviolet curable adhesive, and an anaerobic adhesive can be used.

As the second substrate 721, a flexible organic material such as paper or plastic can be used. Alternatively, as the second substrate 721, a flexible inorganic material may be used. ARTON (manufactured by JSR) formed of poly norbornene having a polar group can be used as a plastic substrate. In addition, polyester typified by polyethylene terephthalate (PET); polyether sulfone (PES); polyethylene naphthalate (PEN); polycarbonate (PC); nylon; polyether etherketone (PEEK); polysulfone (PSF); polyether imide (PEI); polyarylate (PAR); polybutylene terephthalate (PBT); polyimide; an acrylonitrile butadiene styrene resin; poly vinyl chloride; polypropylene; poly vinyl acetate; an acrylic resin; and the like can be given. It is preferable that the second substrate 721 have high thermal conductivity of approximately 2 to 30 W/mK in order to disperse heat generated in an integrated circuit.

Next, as shown in FIG. 12B, after removing the protective layer 711, an adhesive 726 is applied on the third interlayer insulating film 701 so as to cover the antenna 705, whereby a cover material 725 is attached thereto. Similarly to the second substrate 721, a flexible organic material such as paper or plastic can be used as the cover material 725. For example, the thickness of the adhesive 726 may be 10 to 200 μm.

A material which can attach the cover material 725, the third interlayer insulating film 701, and the antenna 705 is used for the adhesive 726. As the adhesive 726, for example, various curable adhesives such as a reactive curable adhesive, a thermal curable adhesive, and a photo curable adhesive such as an ultraviolet curable adhesive, and an anaerobic adhesive can be used.

Through the above-described steps, the ID chip is completed. By the above-described formation method, an integrated circuit having a total thickness of greater than or equal to 0.3 μm and less than or equal to 3 μm, typically approximately 2 μm, which is considerably thin, can be formed between the second substrate 721 and the cover material 725. It is to be noted that the thickness of the integrated circuit includes the thicknesses of various insulating films and interlayer insulating films formed between the adhesive 722 and the adhesive 726 as well as the thickness of a semiconductor element itself. In addition, the integrated circuit included in the ID chip can be formed so as to occupy an area of less than or equal to 5 mm square (25 mm²), more desirably, approximately 0.3 mm square (0.09 mm²) to 4 mm square (16 mm²).

Further, when the integrated circuit is provided at a position between the second substrate 721 and the cover material 725 that is more centered, mechanical strength of the ID chip can be increased. Specifically, when the distance between the second substrate 721 and the cover material 725 is set to be d, it is desirable that the thicknesses of the adhesive 722 and the adhesive 726χ be controlled so that the distance between the second substrate 721 and the center in a direction of the thickness of the integrated circuit satisfies the following Formula 3.

$$\frac{1}{2} d - 30 μ m < χ < \frac{1}{2} d + 30 μ m \qquad [ Formula 3 ]$$

It is further preferable that the thicknesses of the adhesive 722 and the adhesive 726χ be controlled so that the following Formula 4 is satisfied.

$$\frac{1}{2} d - 1 \; 0 μ m < χ < \frac{1}{2} d + 10 μ m \qquad [ Formula 4 ]$$

It is to be noted that the example in which the cover material 725 is used is shown in FIG. 12B; however, the present invention is not limited thereto. For example, the formation may be finished in the step shown in FIG. 12A.

The method in which the separation layer is provided between the first substrate 601 having high heat resistance and the integrated circuit, and the separation layer is removed by etching so that the substrate and the integrated circuit are separated from each other is shown in this embodiment; however, a manufacturing method of the ID chip of the present invention is not limited to this structure. For example, a metal oxide film may be provided between the substrate having high heat resistance and the integrated circuit, and the metal oxide film may be weakened by crystallization, so that the integrated circuit is separated. Alternatively, a separation layer formed of an amorphous semiconductor film containing hydrogen may be provided between the substrate having high heat resistance and the integrated circuit, and the separation layer may be removed by laser light irradiation, so that the substrate and the integrated circuit are separated from each other. Alternatively, the substrate having high heat resistance over which the integrated circuit is formed is mechanically removed or removed by etching with the use of a solution or a gas, so that the integrated circuit may be separated from the substrate.

In the case of using an organic resin for the adhesive 722 which is in contact with the base film 603 in order to ensure flexibility of the ID chip, the use of a silicon nitride film or a silicon oxide film containing nitrogen as the base film 603 makes it possible to prevent an alkali metal such as Na or an alkaline earth metal from diffusing into the semiconductor film, from the organic resin.

In the case where a surface of an object is curved and therefore the second substrate 721 of the ID chip attached to the curved surface is curved so as to have a curved surface along movement of a generating line of a conical surface, a columnar surface, or the like, it is desirable to make a direction of the generating line and a moving direction of carriers of the TFTs 611, 612, and 613 be the same. The above-described structure makes it possible to suppress effects on electric characteristics of the TFTs 611, 612, and 613 even when the second substrate 721 is curved. In addition, when a ratio of an area occupied by the island-shaped semiconductor film in the integrated circuit is set to be 1 to 30%, effects on the electric characteristics of the TFTs 611, 612, and 613 can be further suppressed even when the second substrate 721 is curved.

In this embodiment, the example in which the antenna is formed over the same substrate as the integrated circuit is explained; however, the present invention is not limited to this structure. The antenna and the integrated circuit may be formed over different substrates and attached to each other afterward, so that they are electrically connected to each other.

Further, the frequency of an electric wave generally applied in the ID chip is 13.56 MHz or 2.45 GHz, and it is very important to form the ID chip so that the electric wave with the frequency can be detected, in order to enhance versatility.

The ID chip of this embodiment has a merit in that an electric wave is hardly blocked and a signal can be prevented from attenuating due to the blocked electric wave, in comparison with an ID chip formed using a semiconductor substrate. Therefore, the semiconductor substrate is not necessary to be used, and thus, costs of the ID chip can be drastically reduced. For example, the case of using a silicon substrate of 12 inches in diameter and the case of using a glass substrate with an area of 730 mm×920 mm are compared with each other. An area of the former silicon substrate is about 73000 mm² while an area of the latter glass substrate is about 672000 mm², and therefore, an area of the glass substrate is about 9.2 times as large as that of the case of using the silicon substrate. In the glass substrate with an area of about 672000 mm², about 672000 ID chips of 1 mm square are estimated to be produced if areas which are consumed by division of the substrate are not counted. The number of ID chips is about 9.2 times as large as that of the case of using the silicon substrate. Since the case of using the glass substrate with an area of 730 mm×920 mm needs smaller number of steps than the case of using the silicon substrate of 12 inches in diameter, investment costs in facilities for mass production of the ID chip can be reduced to one-third. Furthermore, according to the present invention, the glass substrate can be reused after separating the integrated circuit therefrom. Therefore, the costs can be drastically reduced in comparison with the case of using the silicon substrate even if costs for covering a cracked glass substrate or cleaning the surface of the glass substrate are taken into consideration. Moreover, if the glass substrate is not reused and is discarded, since the price of the glass substrate with an area of 730 mm×920 mm is about half of the price of the silicon substrate with 12 inches in diameter, the costs of manufacturing the ID chip can be drastically reduced.

Therefore, it is found that, in the case of using the glass substrate with an area of 730 mm×920 mm, the price of the ID chip can be suppressed to about one-thirtieth in comparison with the case of using the silicon substrate of 12 inches in diameter. The use of the ID chip as premises for a disposable one is considered, and thus, the ID chip of the present invention which can be formed with drastically reduced costs is very useful for the above-described purpose.

In this embodiment, the example in which the integrated circuit is separated to be attached to the flexible substrate is explained; however, the present invention is not limited to this structure. For example, in the case of using a substrate having allowable temperature limit which can resist heat treatment in manufacturing steps of the integrated circuit, such as glass substrate, the integrated circuit is not necessarily separated.

This embodiment can be freely combined with Embodiment Mode and other embodiments, if necessary.

### Embodiment 4

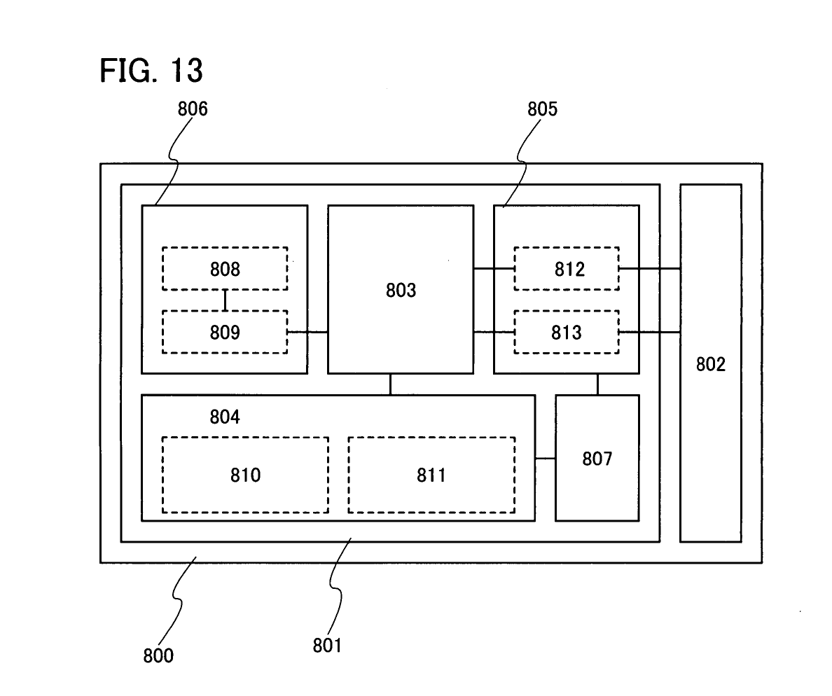

In this embodiment, a structure of an ID chip used for a biological information detection sensor of the present invention and a structure of the sensor will be explained with reference to FIGS. 13, 14, 15, 16, 17, 18, and 19.

FIG. 13 shows a structure of an ID chip 800 provided with an integrated circuit portion 801 and an antenna 802. The integrated circuit portion 801 is provided with a sensor portion (a biological information detection portion) 806 which detects temperature, humidity, illuminance, and other characteristics by physical or chemical means. The sensor portion 806 includes a sensor 808 and a sensor circuit 809 for controlling the sensor 808. The sensor 808 is formed of a semiconductor element such as a resistance element, a capacitively-coupled element, an inductively-coupled element, a photovoltaic element, a photoelectric conversion element, a thermoelectric element, a piezoelectric element, a transistor, a thermistor, or a diode. The structure of the sensor 808 may be changed depending on biological information to be detected. For example, in order to detect pulse, a piezoelectric element may be used. Also, in order to measure body temperature, a thermoelectric element or a resistance element may be used. The sensor circuit 809 detects changes in impedance, reactance, inductance, voltage, or current, and performs analog/digital conversion (A/D conversion) to output a signal to an arithmetic processing circuit portion 803.

A memory portion 804 is provided with one or both of a read-only memory and a rewritable memory. The memory portion 804 is formed of a static RAM, an EEPROM (Electrically Erasable Programmable Read-Only Memory), a flash memory, or the like, whereby external information received through the sensor portion 806 and the antenna 802 can be recorded as needed. The memory portion 804 can be formed of a first memory portion 810 for storing a signal detected by the sensor portion 806 and a second memory portion 811 for recording information written by a reader/writer device. In addition, a read-only memory portion may be formed of a mask ROM or a programmable ROM.

It is preferable that, in order to record biological information detected by the sensor portion 806, the first memory portion 810 be sequentially-writable and be formed by a flash memory in which data is not erased. In addition, it is preferable to apply a once-writable memory element which has a floating gate structure.

A communication circuit portion 805 includes a demodulation circuit 812 and a modulation circuit 813. The demodulation circuit 812 demodulates a signal input through the antenna 802 and outputs the demodulated signal to the arithmetic processing circuit portion 803. The signal includes a signal for controlling the sensor portion 806 and information to be recorded in the memory portion 804. A signal output from the sensor circuit 809 or information read from the memory portion 804 is output to the modulation circuit 813 through the arithmetic processing circuit 803. The modulation circuit 813 modulates this signal to a signal which can be communicated wirelessly and outputs the signal to an external device through the antenna 802.

Power necessary for operating the arithmetic processing circuit portion 803, the sensor portion 806, the memory portion 804, and the communication circuit portion 805 is supplied through the antenna 802. The antenna 802 receives an electromagnetic wave supplied from an external device referred to as a reader/writer, and generates necessary power in a power circuit portion 807. The antenna 802 may be appropriately designed depending on a frequency band for communication. As the frequency band of the electromagnetic wave, a long wave band up to 135 kHz, a short wave band of 6 to 60 MHz (typically, 13.56 MHz), an ultra short wave band of 400 to 950 MHz, a micro wave band of 2 to 25 GHz, or the like can be used. As an antenna for the long wave band or the short wave band, an antenna utilizing electromagnetic induction by a loop antenna is used. Besides, an antenna utilizing mutual induction (electromagnetic coupling type) or electrostatic induction (electrostatic coupling type) may also be used. Power is generated in the power supply circuit portion 807. The antenna 802 may be provided to be divided into a data communication antenna and a power supply antenna.

Such an antenna 802 is formed of a metal material including aluminum, copper, or silver. For example, the antenna 802 can be formed using a paste composition of copper or silver by a printing method such as screen printing, offset printing, or inkjet printing. Alternatively, the antenna 802 may be formed in such a manner that an aluminum film is formed by sputtering or the like and the aluminum film is etched. Besides, the antenna 802 may be formed using an electro plating method or an electroless plating method. Needless to say, the antenna 802 may be formed using a similar material and through a similar formation steps as those of the antenna 477 of Embodiment 2 and the antenna 705 of Embodiment 3.

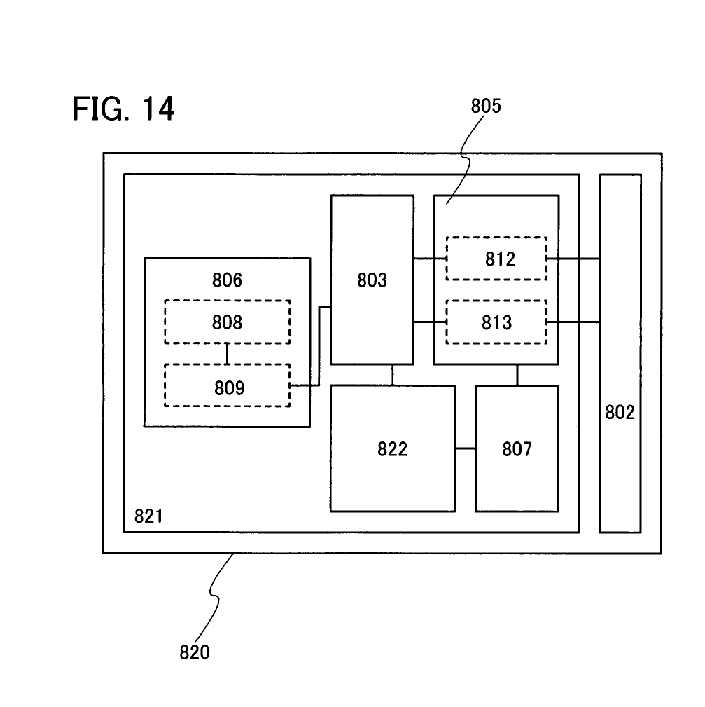

FIG. 14 shows an ID chip 820 in which the structure of the memory portion 804 in the ID chip 800 shown in FIG. 13 is changed. An integrated circuit portion 821 makes a memory portion 822 be sequentially-writable and is formed of a memory element having a floating gate structure in which data is not erased. In particular, it is preferable that a once-writable memory element having a floating gate structure be applied. The ID chip having this structure has only functions of recording and reading data detected by the sensor 808. When the functions are simplified, the ID chip 820 can be downsized. In addition, power can be saved.

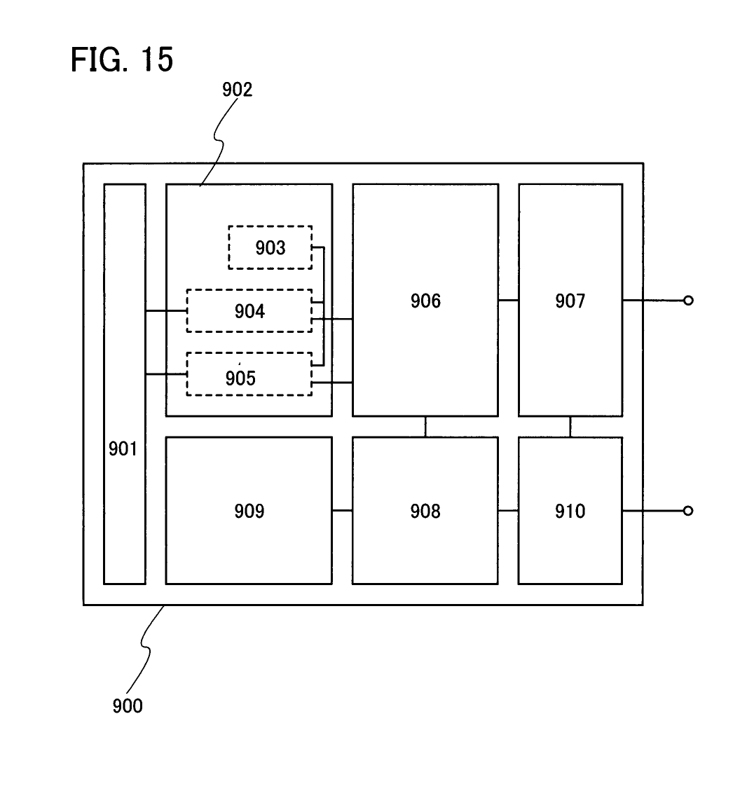

FIG. 15 shows an example of a reader/writer module 900 which sends information to and receives information from the ID chip 800. The reader/writer module 900 is provided with a communication circuit portion 902 including an antenna 901, an oscillator 903, a demodulation circuit 904, and a modulation circuit 905. Besides, the reader/writer module 900 is provided with an arithmetic processing circuit portion 906 and an external interface portion 907 and can be connected to an information processing device such as a computer. In order to encode a control signal for sending and receiving, the reader/writer module 900 may be provided with an encryption/decryption circuit portion 908 and a memory portion 909. A power supply circuit portion 910 supplies power to each circuit.

The ID chip 800 shown in FIG. 13 and the ID chip 820 shown in FIG. 14 are formed by the combination of an active element such as a transistor manufactured of a single crystal semiconductor or a TFT manufactured of a polycrystalline semiconductor film and other passive element. Needless to say, the ID chip may be formed according to the description of Embodiment 2 or Embodiment 3.

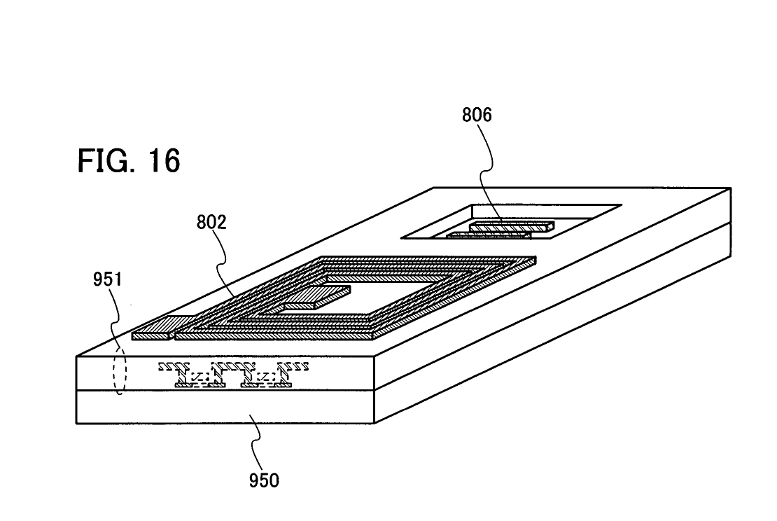

FIG. 16 shows a perspective view of a structural example of the ID chip 800 or the ID chip 820 in which a substrate 950, an element formation layer 951, and the antenna 802 are stacked. The substrate 950 may be similar to the substrate 491 of Embodiment 2 or the substrate 721 of Embodiment 3, for example. In the element formation layer 951, for example, the base film 403, the island-shaped semiconductor films 404, 405, and 406, the gate insulating films 407, 408, and 409, the gate electrodes 441, 442, and 443, the passivation film 461, the interlayer insulating films 462 and 463, and the electrode or wiring 471 shown in FIG. 8B are included. Alternatively, in the element formation layer 951, the base film 603, the island-shaped semiconductor films 621, 622, and 623, the gate insulating films 661, 662, and 663, the sidewalls 665, 666, and 667, the gate electrodes 671, 672, and 673, the passivation film 681, the interlayer insulating films 682 and 683, and the electrodes or wirings 691, 692, 693, 694, and 695 shown in FIG. 12B may be included. The antenna 802 is connected to a circuit formed of a TFT. A protective film formed of an inorganic insulating material or an organic insulating material may be further formed over the antenna 802. When the element formation layer 951 and the antenna 802 are formed together in this manner, downsizing of the ID chip can be achieved. The ID chip 800 or the ID chip 820 is provided with the sensor portion 806. The sensor portion 806 may have a structure in which a light introducing window or an electrode for measuring electrostatic capacitance that is exposed is provided.

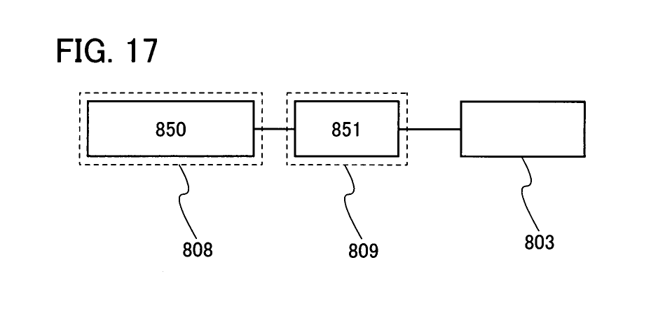

FIG. 17 shows an example of the sensor portion 806. This sensor portion 806 detects temperature. The sensor 808 is formed of plural stages of ring oscillators 850 using a TFT. The sensor 808 utilizes the fact that the oscillation frequency of the ring oscillator 850 changes depending on the temperature. Threshold voltage of the TFT is decreased with increase of the temperature. On-current is increased by decrease of the threshold voltage. The ring oscillator 850 has a characteristic that the higher the on-current of the TFT is, the higher the oscillation frequency gets. With the use of this characteristic, the ring oscillator 850 can be used as a temperature sensor. The oscillation frequency of the ring oscillator 850 can be measured by a pulse counter 851 of the sensor circuit 809. A signal of the pulse counter 851 on its own or after being level-shifted may be input to the arithmetic processing circuit portion 803. Body temperature of the human body can be measured by this sensor detecting temperature.

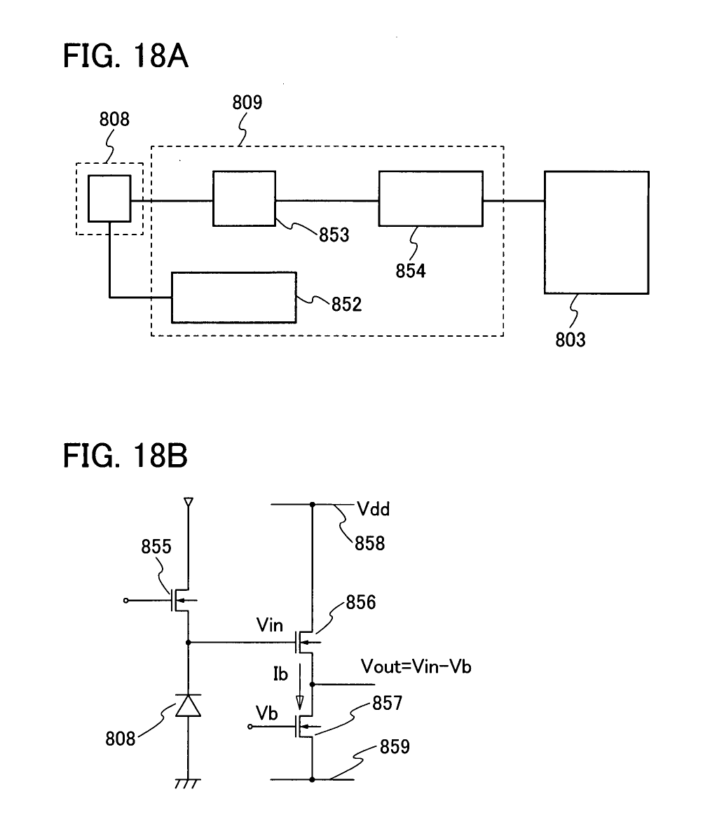

FIG. 18A shows an example of a sensor which detects the presence or absence of light. The sensor 808 is formed of a photodiode, a photo transistor, or the like. The sensor circuit 809 includes a sensor driving portion 852, a detecting portion 853, and an A/D conversion portion 854. For example, time while the human body is exposed to ultraviolet ray can be detected by the photodiode, the photo transistor, or the like.

FIG. 18B is a circuit diagram for explaining the detecting circuit 853. When a reset TFT 855 is made to be in a conduction state, reverse bias voltage is applied to the sensor 808. Here, operation in which potential of a minus terminal of the sensor 808 is charged to potential of power supply voltage is referred to as “reset”. Thereafter, the reset TFT 855 is made to be in non-conduction state. At this time, the potential state is changed with time by an electromotive force of the sensor 808. That is to say, the potential of the minus terminal of the sensor 808 which has been charged to the potential of the power supply voltage is gradually decreased by electric charge generated by photoelectric conversion. When a bias TFT is made to be in a conduction state after a certain period of time has passed, a signal is output to an output side through an amplifying TFT 856. In this case, the amplifying TFT 856 and the bias TFT 857 operate as a so-called source follower circuit.

In FIG. 18B, the example in which the source follower circuit is formed of an n-channel TFT is shown; however, needless to say, the source follower circuit can also be formed of a p-channel TFT. Power supply voltage Vdd is applied to an amplifying side power supply line 858. Reference Potential 0 V is given to a bias side power supply line 859. A drain terminal of the amplifying TFT 856 is connected to the amplifying side power supply line 858, and a source terminal of the amplifying TFT 856 is connected to a drain terminal of the bias TFT 857. A source terminal of the bias TFT 857 is connected to the bias side power supply line 859. Bias voltage Vb is applied to a gate terminal of the bias TFT 857 and bias current Ib flows to this TFT. The bias TFT 857 basically operates as a constant current source. Input voltage Vin is applied to a gate terminal of the amplifying TFT 856, and a source terminal becomes an output terminal. The input-output relation of this source follower circuit is defined as Vout=Vin−Vb. This output voltage Vout is converted into a digital signal by the A/D conversion circuit 854. The digital signal is output to the arithmetic processing circuit portion 803.

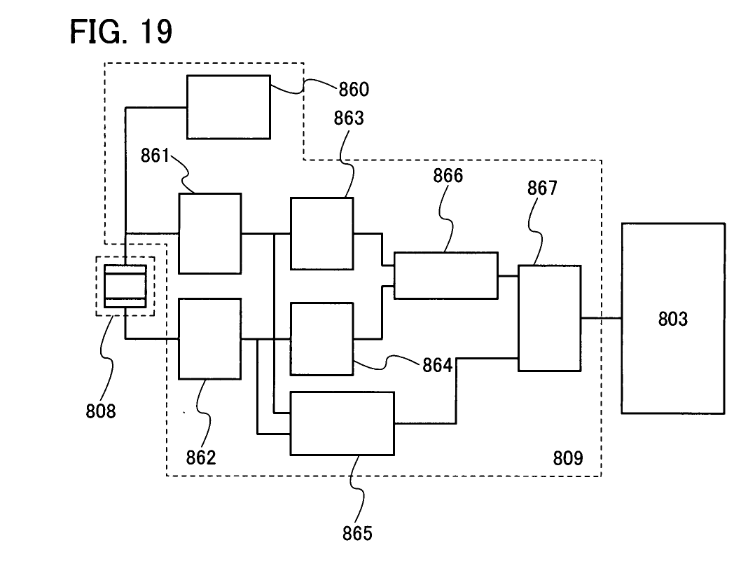

FIG. 19 shows an example in which an element which detects electrostatic capacitance is provided in the sensor 808. The element which detects the electrostatic capacitance is provided with a pair of electrodes. A portion between the electrodes is filled with a medium such as a liquid or a gas. By detection of the change in the electrostatic capacitance between the pair of electrodes, for example, body temperature, pulse, or muscle movement of the human body is detected. In addition, polyimide, acrylic, or other hygroscopic dielectric body is interposed between the pair of electrodes and a small change in electric resistance is read, whereby perspiration of the human body can be detected.

The sensor circuit 809 has a structure shown below. An oscillation circuit (a pulse generator) 860 generates a measurement reference signal and inputs the measurement reference signal to an electrode of the sensor 808. Voltage at this time is input to a voltage detecting circuit 861 as well. A reference signal detected by the voltage detecting circuit 861 is converted into a voltage signal showing an effective value by a conversion circuit 863. Current flowing between electrodes of the sensor 808 is detected by a current detecting circuit 862. A signal detected by the current detecting circuit 862 is converted into a current signal showing an effective value by a conversion circuit 864. Arithmetic processing is performed to the voltage signal which is the output of the conversion circuit 863 and the current signal which is the output of the conversion circuit 864 by an arithmetic circuit 866, so that electric parameter such as impedance or admittance is calculated. In addition, the output of the voltage detecting circuit 861 and the output of the current detecting circuit 862 are input to a phase comparison circuit 865. The phase comparison circuit 865 outputs the phase difference in both signals to an arithmetic circuit 867. The arithmetic circuit 867 calculates electrostatic capacitance with the use of the output signals of the arithmetic circuit 866 and the phase comparison circuit 865. Then, the signal is input to the arithmetic processing circuit portion 803.

Further, the structure in which the sensor portion (the biological information detecting portion) 108 is built in the ID chip is shown in this embodiment; however, a structure may be employed in which the sensor portion is formed separately from the ID chip, which is attached externally.

In addition, this embodiment can be freely combined with Embodiment Mode and other embodiments, if necessary.

This application is based on Japanese Patent Application serial No. 2006-124784 filed in Japan Patent Office on Apr. 28, 2006, the entire contents of which are hereby incorporated by reference.

What is claimed is:

1. A biological information detection sensor device comprising:
an adhesive tape having a first surface which is provided with an adhesive material and a second surface which is not provided with the adhesive material;
a sensor attached on the first surface of the adhesive tape; and
an antenna connected to the sensor,
wherein the sensor comprises a biological information detection portion and a memory portion,
wherein the antenna is pulled from the first surface to the second surface through a groove provided in the adhesive tape, and
wherein the memory portion is capable of storing biological information.

2. The biological information detection sensor device according to claim 1, wherein the antenna is fixed by an adhesive material provided on a bottom surface of the groove.

3. The biological information detection sensor device according to claim 1, wherein the antenna is fixed by a protrusion provided on side surfaces of the groove.

4. The biological information detection sensor device according to claim 1, wherein the sensor further comprises an arithmetic processing circuit portion and a communication circuit portion.

5. The biological information detection sensor device according to claim 1, wherein the biological information detection portion comprises a piezoelectric element or a thermoelectric element.

6. A biological information detection sensor device comprising:
an adhesive tape having a first surface which is provided with an adhesive material and a second surface which is not provided with the adhesive material;
a sensor attached on the first surface of the adhesive tape; and
an antenna connected to the sensor,
wherein the sensor comprises a biological information detection portion and a memory portion each of which is formed of a thin film transistor over an insulating surface,
wherein the antenna is pulled from the first surface to the second surface through a groove provided in the adhesive tape, and
wherein the memory portion is capable of storing biological information.

7. The biological information detection sensor device according to claim 6, wherein the antenna is fixed by an adhesive material provided on a bottom surface of the groove.

8. The biological information detection sensor device according to claim 6, wherein the antenna is fixed by a protrusion provided on side surfaces of the groove.

9. The biological information detection sensor device according to claim 6, wherein the sensor further comprises an arithmetic processing circuit portion and a communication circuit portion.

10. The biological information detection sensor device according to claim 6, wherein the biological information detection portion comprises a piezoelectric element or a thermoelectric element.

11. A biological information detection sensor device comprising:
an adhesive tape having a first surface which is provided with an adhesive material and a second surface which is not provided with the adhesive material;
a sensor attached on the first surface of the adhesive tape; and
an antenna connected to the sensor,
wherein the sensor comprises a biological information detection portion and an ID chip,
wherein the antenna is pulled from the first surface of the adhesive tape to the second surface through a groove provided in the adhesive tape, and
wherein the ID chip is capable of storing biological information.

12. The biological information detection sensor device according to claim 11, wherein information stored in the ID chip and information detected by the sensor are sent and received by the antenna.

13. The biological information detection sensor device according to claim 11, wherein the biological information detection portion comprises a piezoelectric element or a thermoelectric element.

14. The biological information detection sensor device according to claim 11,
wherein the antenna is fixed to the second surface of the adhesive tape by a fixation mechanism provided on the second surface of the adhesive tape.

15. The biological information detection sensor device according to claim 14,
wherein the fixation mechanism is a mechanism in which a groove is provided on the second surface of the adhesive tape and an adhesive material is provided on a bottom surface of the groove so that the antenna is fixed.

16. The biological information detection sensor device according to claim 14,
wherein the fixation mechanism is a mechanism in which a groove is provided on the second surface of the adhesive tape and a protrusion is provided on side surfaces of the groove so that the antenna is fixed.
